(19) United States
(12) Patent Application Publication
(10) Pub. No.: US 2011/0045528 A1
(43) Pub. Date: Feb. 24, 2011

(54) APPARATUS AND METHOD FOR ENHANCED DISRUPTION AND EXTRACTION OF INTRACELLULAR MATERIALS FROM MICROBIAL CELLS

(76) Inventor: Srisuda Dhamwichukorn, Edmonds, WA (US)

Correspondence Address: DAVIS WRIGHT TREMAINE, LLP/Seattle, 1201 Third Avenue, Suite 2200, SEATTLE, WA, 98101-3045, US

(21) Appl. No.: 12/852,403

(22) Filed: Aug. 6, 2010

Related U.S. Application Data
(60) Provisional application No. 61/238,077, filed on Aug. 28, 2009.
(60) Provisional application No. 61/235,655, filed on Aug. 20, 2009.

(51) Int. Cl. C12P 1/00; C12N 1/00; C12N 1/12; C12N 1/20; C12N 1/14; C12N 1/16; C12N 1/06

(52) U.S. Cl. 435/41; 435/243; 435/257.1; 435/252.1; 435/254.1; 435/255.1; 435/259

(57) ABSTRACT

Provided are surprisingly beneficial osmotic stress shock methods for facilitating disruption and/or extraction of microorganisms, comprising a synergistic combination of at least one hypotonic shock and at least two hypertonic shocks, wherein the last shock is a hypertonic shock. Preferred aspects of the invention relate to facilitating disruption of a broad spectrum of microorganisms (e.g., algae, bacteria, yeast, fungus, etc.). In particular aspects the microbial cells are subjected to: a hypertonic/hypotonic/hypertonic tertiary shock; a hypotonic/hypertonic/hypertonic tertiary shock; or a hypotonic/hypertonic/hypotonic/hypertonic quaternary shock. Particular aspects further comprise disrupting and/or extracting of the shocked microbial cells, and isolating a cellular constituent or bioproduct (e.g., biofuel, biocrude, bioenergy, biogas, biodiesel, bioethanol, biogasoline, pharmaceuticals, nutraceuticals, food, vitamins, feedstock, dyes, colorants, sulfur, fertilizer, bioplastic) therefrom. In particular preferred aspects, facilitation of algae disruption and/or extraction is provided and in certain embodiments, isolation of at least one of lipid, oil, and triacylglycerol is enhanced.

### CROSS-REFERENCE TO RELATED APPLICATIONS

This application claims the benefit of priority to U.S. Provisional Patent Application Ser. Nos. 61/238,077, filed 28 Aug. 2009, entitled “Apparatus and Method for Enhancing Disruption and Extraction of Intracellular Materials from Microbial Cells,” and 61/235,655, filed 20 Aug. 2009, entitled “Method for Enhanced Sustainable Production of Algal Bio-Products, Comprising Use of Symbiotic Diazotroph-Attenuated Stress Co-Cultivation,” which are incorporated herein by reference in their entirety.

### FIELD OF THE INVENTION

Particular aspects relate generally to disruption, extraction, and recovery of products from microbial organisms, and more particularly to novel apparatus and methods for osmotically treating microbial organisms (e.g., algae, bacteria, yeast, fungus, etc.) to facilitate subsequent disruption and/or extraction to enhance recovery of products (e.g., cellular and intracellular products. Particular preferred aspects relate to a surprisingly beneficial synergistic osmotic stress protocol comprising at least one hypotonic shock and at least two hypertonic shocks, wherein the last shock of the protocol is a hypertonic shock, wherein enhancing at least one of disruption and extraction of microbial cells is afforded.

### BACKGROUND

Microbial cells are important resources for many beneficial bio-products. For example, algae cells contain pigments and other intracellular matters for nutraceuticals, vitamins, bioplastics, dyes and colorants, feedstock, pharmaceuticals, algae fuel and especially oils for energy and health care purposes. In addition, algal, fungal, and yeast cells contain proteins, carbohydrates and fatty acids or oil. Proteins can be used as protein supplements or feedstock. Carbohydrates can be used for biogas and bioethanol production. Oil and fatty acids can be used as biocrude or oil for biodiesel production. In addition, pigments, oils or many intracellular materials, biochemicals, metabolites and recombinant-based or recombinant-mediated products can be used for pharmaceuticals, nutraceuticals and/or cosmeceuticals.

Harvesting and extracting microbial cells can, however, be costly and time consuming. To extract bio-products from microbial cells, the cell walls must be disrupted and the bio-product extracted from the total cell lysis product, preferably without appreciable loss and/or degradation of the product sought. The microbial cell walls of algae and fungi are, moreover, inherently difficult to disrupt by conventional means alone. Furthermore, some methods of cell disruption are effective at a laboratory scale, but not economical at a commercial scale. For example, mechanical screw press methods are not efficient, and extract relatively little oil from microbial cells. Ultrasonic technology, on the other hand, requires substantial power use to ensure complete cell disruption and, typically requires additional power consumption to control temperature due to heat generation, and therefore, is much more expensive than, for example hexane extraction. Likewise, hexane extraction, particularly in methods using solely hexane as the disrupting agent, is not only environmentally hostile because hexane is an organic chemical pollutant, but is also energy intensive because of energy use inherent in manufacturing of hexane. Moreover, typical prior art commercial microbial extraction methods rely on the use of expensive equipment and large amounts of hexane required to obtain appropriate yields of bio-products.

There is, therefore, from both environmental and economic perspectives, a pronounced need in the art for novel compositions and methods to provide for robust microbial extraction methods, but with less reliance on expensive, energy intensive equipment and processes, and large amounts of chemical pollutants (e.g., hexane) to obtain adequate yields of bio-products.

### SUMMARY OF THE INVENTION

Particular aspects provide a surprisingly beneficial synergistic osmotic stress protocol for facilitating disruption and/or extraction of microorganisms, comprising at least one hypotonic shock and at least two hypertonic shocks, wherein the last shock is a hypertonic shock. While preferred aspects of the invention relate to facilitation of algal cell disruption, the synergistic methods are applicable to facilitating disruption of a broad spectrum of microorganisms (e.g., algae, bacteria, yeast, fungus, etc.).

As appreciated in the art, there are interactions between the cell wall and the cell membrane of plants. For example, in plant cells suspended in a medium, the terms isotonic, hypotonic and hypertonic, which relate to solutes separated by a membrane, cannot strictly be used accurately because the pressure exerted by the cell wall significantly affects the osmotic equilibrium point. Additionally, as further appreciated in the art, a hypertonic environment forces water to leave a cell, such that in plant cells for example, in a process referred to as plasmolysis, the flexible cell membrane pulls away from the rigid cell wall, but remains joined, in pincushion fashion, to the cell wall at points called plasmodesmata, which become constricted and largely cease to function.

According to particular aspects of the present invention, the Applicant has discovered a unique synergistic method to productively destabilize, and thereby exploit, the apparent stabilizing interactions between the cellular membrane and the cell wall of plant cells. According to certain aspects, disruption of plant cells is facilitated by subjecting the plant cell wall to hypotonic shock after exposure of the cells to a hypertonic or isotonic medium, and imposing at least one final hypertonic shock. Without being bound by mechanism, according to particular aspects, the disruption of plant cells is facilitated by synergistically destabilizing the interaction between the cell wall and the cell membrane, such that the cell wall is more susceptible to shock-mediated weakening (e.g., hypotonic shock-mediated weakening).

In particular aspects the microbial cells are subjected to: a synergistic hypertonic/hypotonic/hypertonic tertiary shock; a synergistic hypotonic/hypertonic/hypertonic tertiary shock; or a synergistic hypotonic/hypertonic/hypotonic/hypertonic quaternary shock. Particular aspects further comprise disrupting and/or extracting of the synergistically shocked microbial cells, and enhanced isolation of a cellular constituent or material for bioproducts (e.g., biofuel, biocrude, bioenergy, biogas, biodiesel, bioethanol, biogasoline, pharmaceuticals, nutraceuticals, food, vitamins, feedstock, dyes, colorants, sulfur, fertilizer, bioplastic) therefrom. In particular preferred aspects, synergistic facilitation of algae disruption and/or extraction is provided and in certain embodiments, at least one of lipid, oil, and triacylglycerol is isolated therefrom.

Particular aspects provide methods for enhancing disruption or extraction of microbial cells, comprising: obtaining microbial cells, the microbial cells having a cell membrane and having been grown in a source medium having a source tonicity with respect to said membrane; suspending the microbial cells, for a primary shock time period, in an initial aqueous suspension medium having a primary tonicity different from the source tonicity to provide primary shocked microbial cells; suspending the primary shocked microbial cells, for a secondary shock time period, in an secondary aqueous suspension medium having a secondary tonicity different from the primary tonicity to provide secondary shocked microbial cells; suspending the secondary shocked microbial cells, for a tertiary shock time period, in an tertiary aqueous suspension medium having a tertiary tonicity that is the same or different from the secondary tonicity to provide tertiary shocked microbial cells; and subjecting the tertiary shocked microbial cells to a suitable disruption method, wherein the tonicity shocking comprises a synergistic combination of at least one hypotonic shock and at least two hypertonic shocks, and wherein the last tonicity shock is a hypertonic shock, wherein enhancing at least one of disruption and extraction of microbial cells is afforded. In certain aspects, the source medium is fresh water having a fresh water tonicity, the primary tonicity is hypertonic, and the microbial cells are subjected to a hypertonic/hypotonic/hypertonic tertiary shock. In alternate embodiments, the source medium is saltwater, brackish water, or marine water, having a salt, brackish or marine water tonicity, respectively, the primary tonicity is hypotonic, and the microbial cells are subjected to a hypotonic/hypertonic/hypertonic tertiary shock.

Particular aspects of the method further comprise, after the tertiary shock time period and prior to subjecting to a suitable disruption period, suspending the tertiary shocked microbial cells, for a quaternary shock time period, in an quaternary aqueous suspension medium having a quaternary tonicity that is different from the tertiary tonicity to provide quaternary shocked microbial cells; and subjecting the quaternary shocked microbial cells to a suitable disruption method. In certain aspects, the source medium is saltwater, brackish water, or marine water, having a salt, brackish or marine water tonicity, respectively, the primary tonicity is hypotonic, and the microbial cells are subjected to a hypotonic/hypertonic/hypotonic/hypertonic quaternary shock.

In particular embodiments of the methods, at least one hypotonic shock comprises a hypotonic shock equivalent to at least 5 g/L total dissolved salt/solids (TDS), and wherein at least one of the at least two hypertonic shocks comprises a hypertonic shock equivalent to at least 15 g/L TDS. In certain embodiments, the at least one hypotonic shock comprises a hypotonic shock equivalent to a value in the range of from about 8 g/L to about 12 g/L total dissolved salt (TDS), and at least one of the at least two hypertonic shocks comprises a hypertonic shock in the range of from about 15 g/L to about 60 g/L total dissolved salt (TDS). In particular aspects, the at least one hypotonic shock comprises a hypotonic shock equivalent to a value in the range of from about 9 g/L to about 10 g/L total dissolved salt (TDS), and at least one of the at least two hypertonic shocks comprises a hypertonic shock in the range of from about 20 g/L to about 35 g/L total dissolved salt (TDS).

In certain aspects of the methods, at least one of suspending the microbial cells, suspending the primary shocked microbial cells, and suspending the secondary shocked microbial cells comprises at least one of: addition of saline to the suspension medium; changing the suspension medium; and diluting the suspension medium. In certain aspects of the methods, at least one of suspending the microbial cells, suspending the primary shocked microbial cells, suspending the secondary shocked microbial cells, and suspending the tertiary shocked microbial cells, comprises at least one of: addition of saline to the suspension medium; changing the suspension medium; and diluting the suspension medium.

In certain aspects of the methods, the primary shock time period, the secondary shock time period, the tertiary shock time period, and the quaternary shock time period are equal or substantially equal in duration (e.g., said duration being a duration in the range of about 0.5 hr to about 4 hr. In certain aspects of the methods, the duration of at least two of the primary shock time period, the secondary shock time period, the tertiary shock time period, and the quaternary shock time period differ. In particular embodiments, the duration of the at least one hypotonic shock is less than the duration of at least one of the at least two hypertonic shocks. In particular aspects, the duration of the at least one hypotonic shock is: less than or equal to about 30 minutes; in a range of about 2 minutes to about 30 minutes; or in a range of about 5 minutes to about 20 minutes. In certain embodiments, the duration of at least one of the at least two hypertonic shocks is in a range of about 2 minutes to about 30 minutes.

In certain aspects of the methods, the microorganism comprises a microorganism selected from the group consisting of algae, bacteria, yeast, and fungus. In particular embodiments, the microorganism comprises algae.

Further aspects of the invention provide methods for enhancing disruption or extraction of algae, comprising: obtaining algal cells, the algal cells having a cell membrane and having been grown in a source medium having a source tonicity with respect to said membrane; suspending the algal cells, for a primary shock time period, in an initial aqueous suspension medium having a primary tonicity different from the source tonicity to provide primary shocked algal cells; suspending the primary shocked algal cells, for a secondary shock time period, in an secondary aqueous suspension medium having a secondary tonicity different from the primary tonicity to provide secondary shocked algal cells; suspending the secondary shocked algal cells, for a tertiary shock time period, in an tertiary aqueous suspension medium having a tertiary tonicity that is the same or different from the secondary tonicity to provide tertiary shocked algal cells; and subjecting the tertiary shocked algal cells to a suitable disruption method, wherein the tonicity shocking comprises a synergistic combination of at least one hypotonic shock and at least two hypertonic shocks, and wherein the last tonicity shock is a hypertonic shock, wherein enhancing at least one of disruption and extraction of algal cells is afforded.

Yet further aspects provide methods for enhancing disruption or extraction of algae, comprising: obtaining algal cells, the algal cells having a cell membrane and having been grown in a source medium having a source tonicity with respect to said membrane; suspending the algal cells, for a primary shock time period, in an initial aqueous suspension medium having a primary tonicity different from the source tonicity to provide primary shocked algal cells; suspending the primary shocked algal cells, for a secondary shock time period, in an secondary aqueous suspension medium having a secondary tonicity different from the primary tonicity to provide secondary shocked algal cells; suspending the secondary shocked algal cells, for a tertiary shock time period, in an tertiary aqueous suspension medium having a tertiary tonicity that is the same or different from the secondary tonicity to provide tertiary shocked algal cells; suspending the tertiary shocked microbial cells, for a quaternary shock time period, in an quaternary aqueous suspension medium having a quaternary tonicity that is different from the tertiary tonicity to provide quaternary shocked microbial cells; and subjecting the quaternary shocked algal cells to a suitable disruption method, wherein the tonicity shocking comprises a synergistic combination of at least one hypotonic shock and at least two hypertonic shocks, and wherein the last tonicity shock is a hypertonic shock, wherein enhancing at least one of disruption and extraction of algal cells is afforded.

In particular embodiments of the methods, the algae comprises at least one algal type selected from freshwater, brackish water, saltwater, and marine water algae.

In certain aspects of the methods, the at least one hypotonic shock is of a duration less than or equal to 30 minutes.

In certain aspects of the methods, a temperature below ambient temperature is used during the at least one hypotonic shock and/or during at least one of the at least two hypertonic shock steps. In particular embodiments, a temperature between about 0° C. and about 25° C. is used during the at least one hypotonic shock and/or during at least one of the at least two hypertonic shock steps.

In certain aspects of the methods, the microbial cells comprise both algae and bacteria, and the bacterial are differentially lysed during the synergistic osmotic shock steps. In certain aspects of the methods, the algal cells are present in combination with bacterial cells, and the bacterial are differentially lysed during the synergistic osmotic shock steps.

In particular aspects, the above methods further comprise disrupting and/or extracting of the shocked microbial cells, and may further comprise isolating of a cellular constituent or bioproduct from the disrupted and/or extracted microbial cells.

### BRIEF DESCRIPTION OF THE DRAWINGS

FIG. 1 is a simplified section view of an exemplary apparatus of the invention.

FIG. 2 is a schematic illustration of an exemplary synergistic process for enhancement and extraction of intracellular material from microbial cells (e.g., algae, bacteria, yeast, fungus) using exemplary apparatus of the invention.

FIG. 3 shows an exemplary method, in which fresh water algae or microorganisms are exposed to synergistic, alternating hypertonic and hypotonic solutions, comprising at least one hypotonic shock and at least two hypertonic shocks, wherein the last shock of the protocol is a hypertonic shock; that is, hypertonic/hypotonic/hypertonic.

FIG. 4 shows an exemplary method in which brackish/marine/salt water algae or microorganisms are synergistically exposed to a hypotonic solution, followed by a hypertonic solution, followed by another hypotonic solution, and finally treated with a hypertonic solution; that is, hypotonic/hypertonic/hypotonic/hypertonic.

FIG. 5 shows an exemplary method in which fresh water algae or microorganisms are exposed to a hypertonic solution, followed by a hypotonic solution, followed by an additional hypotonic solution; that is, hypertonic/hypotonic/hypotonic.

FIG. 6 shows an exemplary method in which brackish/marine/saltwater algae or microorganisms are synergistically exposed to a hypotonic solution, followed by a hypertonic solution, and finally treated with a additional hypertonic solution; that is hypotonic/hypertonic/hypertonic.

FIG. 7 shows a graph illustrating temporal aspects of the percent enhanced extraction achieved when the duration of the hypotonic incubation is varied.

### DETAILED DESCRIPTION OF THE INVENTION

Particular aspects provide methods for synergistic, osmotically enhanced disruption and/or extraction of microorganisms to provide bio-products from microbial cells, and in particular aspects provide compositions and methods for synergistically exposing microorganisms to alternating hypertonic and hypotonic shock protocols for surprisingly enhanced disruption and/or extraction of bio-products from microbial cells. Particular aspects further provide methods for enhanced disruption and/or extraction of microorganisms to provide bio-products from fresh water microorganisms, wherein the fresh water microorganisms are exposed to a synergistic hypertonic-hypotonic-hypertonic shock protocol. Particular less preferred aspects provide methods for enhanced disruption and/or extraction of microorganisms to provide bio-products from fresh water microorganisms, wherein the fresh water microorganisms are exposed to a hypertonic-hypotonic-hypotonic shock protocol. Additional aspects provide methods for enhanced disruption and/or extraction of microorganisms to provide bio-products from brackish/marine/saltwater microorganisms, wherein the brackish/marine/saltwater microorganisms are exposed to a synergistic hypotonic-hypertonic-hypertonic shock protocol. Further aspects provide for enhanced disruption and/or extraction of microorganisms to provide bio-products from brackish/marine/saltwater microorganisms, wherein the brackish/marine/saltwater microorganisms are exposed to a synergistic hypotonic-hypertonic-hypotonic-hypertonic shock protocol.

Certain aspects of the invention are directed to enhanced disruption and/or extraction of microorganisms to provide bio-products that are retrieved from microbial cells grown according to the method disclosed in Applicant's U.S. Provisional Patent Application No. 61/235,655, (now U.S. patent application Ser. No. ______, already incorporated herein by reference in its entirety, including for its teachings on synergistic microbial growth, including under nitrogen stress conditions, and comprising diazotroph-attenuated stress growth methods). Further aspects are directed to enhanced disruption and/or extraction of microorganisms to provide bio-products that are retrieved from microbial cultures grown under symbiotic co-cultivation conditions, wherein the symbiotic co-cultivation is comprised of at least one algal species with at least one aerobic bacterial species and at least one diazotroph. Certain aspects are directed to enhanced disruption and/or extraction of microorganisms to provide bio-products that are retrieved from microbial cultures grown under symbiotic co-cultivation conditions, wherein the symbiotic co-cultivation is comprised of at least one algae with at least one diazotroph.

Table 1 shows exemplary preferred organisms for use in producing bio-products using the disclosed synergistic osmotic shock methods to enhance disruption and/or extraction of microorganisms to provide the bio-products.

Table 2 shows exemplary organisms that can be used for a sustainable continuous co-culture of algae, wherein at least one alga is cultivated with at least one aerobic bacteria and at least one diazotroph, and wherein the disclosed synergistic osmotic shock methods are used to enhance disruption and/or extraction of microorganisms to provide the bio-products.

TABLE 1
Exemplar Phyla and Genera of Algae and Fungi and their
Exemplar Species including Identified and Unidentified Species.

| | | |
|---|---|---|
| Phyla | Genera | Exemplar Species |

Algae

| | | |
|---|---|---|
| Chlorophyta | Chlorella | Chlorella vulgaris, Chlorella |
| | | ellipsoidea, Chlorella |
| | | pyrenoidosa, Chlorella spp |
| | Scenedesmus | Scenedesmus Acuminatus, |
| | | Scenedesmus obliquus, |
| | | Scenedesmus quadricauda, |
| | | Scenedesmus dimorphus, |
| | | Scenedesmus spp. |
| | Chlamydomonas | Chlamydomonas rheinhardii, |
| | | Chlamydomonas globosa, |
| | | Chlamydomonas angulosa, |
| | | Chlamydomonas spp. |
| | Spirogyra | Spirogyra neglecta, Spirogyra |
| | | gracilis, Spirogyra spp. |
| Euglenophyta | Euglena: | Euglena rostrifera, Euglena |
| | | gracilis, Euglena spiroides, |
| | | Euglena anabaena, Euglena |
| | | spp. |
| Bacillariophyta | Navicula | Navicula cancellata, Navicula |
| | | menisculus, Navicula |
| | | perminuta, Navicula spp. |
| | Aulacoseira | Aulacoseira islandrica, |
| | | Aulacoseira muszanensis, |
| | | Aulacoseira alpigena, |
| | | Aulacoseira spp. |
| Microspora | Microspora | Microspora floccosa, |
| | | Microspora spp. |
| | Batrachospermum | Batrachospermum turfosum |
| | | Batrachospermum |
| | | gelatinosum, |
| | | Batrachospermum spp. |
| Rhodophyta | Compsopogonopsis | Compsopogonopsis fruticosa, |
| | | Compsopogon minutes, |
| | | Compsogogon spp. |
| | Audoouinella | Audouinella glomerata, |
| | | Audouinella cylindrical, |
| | | Audouinella spp. |

Fungi

| | | |
|---|---|---|
| Ascomycota | Aspergillus | Aspergillus niger, Aspergillus |
| | | nidulans, Aspergillus spp. |
| | Saccharomyces | Saccharomyces cerevisiae, |
| | | Saccharomyces bayanus, |
| | | Saccharomyces boulardii, |
| | | Saccharomyces bulderi, |
| | | Saccharomyces cariocanus, |
| | | Saccharomyces cariocus, |
| | | Saccharomyces chevalieri, |
| | | Saccharomyces dairenensis, |
| | | Saccharomyces ellipsoideus, |
| | | Saccharomyces martiniae, |
| | | Saccharomyces monacensis, |
| | | Saccharomyces norbensis, |
| | | Saccharomyces paradoxus, |
| | | Saccharomyces pastorianus, |
| | | Saccharomyces spencerorum, |
| | | Saccharomyces turicensis, |
| | | Saccharomyces unisporus, |
| | | Saccharomyces uvarum, |
| | | Saccharomyces zonatus, |
| | | Saccharomyces spp. |
| | Fusarium | Fusarium culmorum, |
| | | Fusarium solani, Fusarium |
| | | oxysporum, Fusarium |
| | | chlamydosporum Fusarium |
| | | spp |
| | Xylaria | Xylaria hypoxylon, Xylaria |
| | | polymorpha, Xylaria spp. |
| | Gliocladium | Gliocladium penicilloides, |
| | | Gliocladium virens, |
| | | Gliocladium deliquescens, |
| | | Gliocladium roseum, |
| | | Gliocladium spp. |
| Basidiomycota | Trametes | Trametes versicolor, Trametes |
| | | hirsutus, Trametes spp. |
| | Pleurotus | Pleurotus ostreatus, Pleurotus |
| | | acerosus, Pleurotus australis |
| | | Pleurotus spp. |
| | Phanerochaete | Phanerochaete allantospora, |
| | | Phanerochaete arizonica, |
| | | Phanerochaete avellanea, |
| | | Phanerochaete burtii, |
| | | Phanerochaete carnosa, |
| | | Phanerochaete chrysorhizon, |
| | | Phanerochaete |
| | | chrysosporium, |
| | | Phanerochaete radicata, |
| | | Phanerochaete sordid, |
| | | Phanerochaete spp. |
| | Armillariella | Armillariella mellea, |
| | | Armillariella spp. |
| | Coriolus | Coriolus versicolor, Coriolus |
| | | spp. |
| | Schizophyllum | Schizophyllum commune, |
| | | Schizophyllum spp. |
| | Serpula | Serpula lacrymans, Serpula |
| | | himantioides, Serpula spp. |
| Zygomycota | Mortierella | Mortierella alpina, |
| | | Mortierella spp. |

Certain aspects are directed to enhanced disruption and/or extraction of microorganisms to provide bio-products from microorganisms including but not limited to an algae species. In preferred embodiments, the algae species include but are not limited to marine, brackish water, saltwater, and freshwater algae. In further aspects, the algae species include but are not limited to those species that are derived from acidic or alkaline water. According to particular aspects, the algae species include any micro or macro algal species, including but not limited to, any eukaryotic algae such as diatoms and green, red, and brown algae (e.g., kelp). In particular aspects, the algal and fungal species include but are not limited to those exemplary phyla, genera, and species listed in Table 1.

Exemplary algal species that can be used with the disclosed synergistic methods to enhance production of bio-products include, but are not limited to, Chlorella vulgaris, Chlorella ellipsoidea, Chlorella pyrenoidosa, Chlorella spp, Scenedesmus Acuminatus, Scenedesmus obliquus, Scenedesmus quadricauda, Scenedesmus dimorphus, Scenedesmus spp., Chlamydomonas rheinhardii, Chlamydomonas globosa, Chlamydomonas angulosa, Chlamydomonas spp., Spirogyra neglecta, Spirogyra gracilis, Spirogyra spp, Euglena rostrifera, Euglena gracilis, Euglena spiroides, Euglena anabaena, Euglena spp., Navicula cancellata, Navicula menisculus, Navicula perminuta, Navicula spp., Aulacoseira islandrica, Aulacoseira muszanensis, Aulacoseira alpigena, Aulacoseira spp., Microspora floccosa, Microspora spp., Batrachospermum turfosum Batrachospermum gelatinosum, Batrachospermum spp., Compsopogonopsis fruticosa, Compsopogon minutes, Compsogogon spp., Audouinella glomerata, Audouinella cylindrical, and Audouinella spp.

Exemplary fungal species that can be used with the disclosed synergistic methods to enhance production of bio-products include, but are not limited to, Aspergillus niger, Aspergillus nidulans, Aspergillus spp., Saccharomyces cerevisiae, Saccharomyces bayanus, Saccharomyces boulardii, Saccharomyces bulderi, Saccharomyces cariocanus, Saccharomyces cariocus, Saccharomyces chevalieri, Saccharomyces dairenensis, Saccharomyces ellipsoideus, Saccharomyces martiniae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, Saccharomyces zonatus, Saccharomyces spp., Fusarium culmorum, Fusarium solani, Fusarium oxysporum, Fusarium chlamydosporum, Fusarium spp., Xylaria hypoxylon, Xylaria polymorpha, Xylaria spp., Gliocladium penicilloides, Gliocladium virens, Gliocladium deliquescens, Gliocladium roseum, Gliocladium spp., Trametes versicolor, Trametes hirsutus, Trametes spp., Pleurotus ostreatus, Pleurotus acerosus, Pleurotus australis Pleurotus spp., Phanerochaete allantospora, Phanerochaete arizonica, Phanerochaete avellanea, Phanerochaete burtii, Phanerochaete carnosa, Phanerochaete chrysorhizon, Phanerochaete chrysosporium, Phanerochaete radicata, Phanerochaete sordid, Phanerochaete spp., Armillariella mellea, Armillariella spp., Coriolus versicolor, Coriolus spp., Schizophyllum commune, Schizophyllum spp., Serpula lacrymans, Serpula himantioides, Serpula spp., Mortierella alpine, Mortierella spp.

Exemplary aerobic bacterial species that can be used with the disclosed synergistic methods to enhance production of bio-products bio-products include, but are not limited to those from the classes of Gammaproteobacteria (e.g., Escherichia, Pseudomonas), Actinobacteria (e.g., Rhodococcus), Bacilli, (e.g., Bacillus) and, Beta Proteobacteria (e.g., Achromobacter). and Alphaproteobacteria (e.g., Rhodobacter).

Exemplary diazotrophic species that can be used with the disclosed synergistic methods to enhance production of bio-products bio-products include, but are not limited to, Anabaena Siamensis, Anabaena spiroides, Anabaena cylindrical, Anabaena spp, Spirulina Platensis, Spirulina maxima, Spirulina spp, Calothrix marchica, Calothrix spp., Lyngbya perelegans, Lyngbya wollei, Lyngbya spp., Hapalosiphon Hybernicus, Hapalosiphon spp., Nostoc linckia, Nostoc commune, Nostoc spp., Oscillatoria borneti, Oscillatoria limosa, Oscillatoria princeps, Oscillatoria salina, Oscillatoria okeni, Oscillatoria spp, Gloeocapsa gelatinosa, Gloeocapsa spp., Microcoleu Chthonoplates, Microcoleus spp., Aphanothece stagnina, Aphanothece clathrata, Aphanothece granulosa, Aphanothece spp., Klebsiella pneumonia, Klebsiella spp., Bacillus polymyxa, Bacillus macerans, Bacillus spp., Escherichia intermedia, Escherichia spp., Paenibacillus polymyxa, Paenibacillus macerans, Paenibacillus spp., Azobacter vinelandii, Azobacter spp., Rhodobacter sphaeroides, Rhodobacter capsulatus, Rhodobacter spp., Rhodopseudomonas palustris, Rhodopseudomonas spp., Methanosarcina barkeri, Methanosarcina spp., Methanospirillum hungateii, Methanospirillum spp., Methanobacterium bryantii, and Methanobacterium spp.

TABLE 2
Exemplary Growth Combinations of Algae, Aerobic Bacteria, and Diazotrophs.

| | | Diazotrophs | |
|---|---|---|---|
| | | | Additional Nitrogen |
| Algal Phyla and | Aerobic Bacterial | Nitrogen Fixing | Fixing Bacteria and |
| Genera | Classes | Cyanobacteria | Archaea |
| Chlorophyta: | Gammaproteobacteria | Anabaena, | Klebsiella, |
| Chlorella, | (e.g. Escherichia, | Nostoc, | Bacillus, |
| Scenedesmus, | Pseudomonas) | Spirulina, | Escherichia, |
| Chlamydomonas, | Actinobacteria (e.g. | Synechococcus, | Paenibacillus, |
| Closterium, | Rhodococcus) | Oscillatoria, | Azobacter, |
| Synedra, | Bacillus | Synechocystis, | Rhodobacter, |
| Pediastrum, | Clostridium | Gloeocapsa, | Rhodopseudomonas, |
| Ankistrodesmus, | Beta Proteobacteria (e.g. | Hapalosiphon, | Methanosarcina, |
| Planktosphaeria, | Achromobacter) | Stigonema, | Methanospirillum, |
| Mougeotia | | Microcoleus, | Methanobacterium |
| | | Aphanothece | |
| Euglenophyta: | Gammaproteobacteria | Anabaena, | Klebsiella, |
| Euglena, | (e.g. Escherichia, | Nostoc, | Bacillus, |
| | Pseudomonas) | Spirulina, | Escherichia, |
| | Actinobacteria (e.g. | Synechococcus, | Paenibacillus, |
| | Rhodococcus) | Oscillatoria, | Azobacter, |
| | Bacillus | Synechocystis, | Rhodobacter, |
| | Clostridium | Gloeocapsa, | Rhodopseudomonas, |
| | Beta Proteobacteria (e.g. | Hapalosiphon, | Methanosarcina, |
| | Achromobacter) | Stigonema, | Methanospirillum, |
| | | Microcoleus, | Methanobacterium |
| | | Aphanothece | |
| Bacillariophyta: | Gammaproteobacteria | Anabaena, | Klebsiella, |
| Navicula, | (e.g. Escherichia, | Nostoc, | Bacillus, |
| Surirella | Pseudomonas) | Spirulina, | Escherichia, |
| | Actinobacteria (e.g. | Synechococcus, | Paenibacillus, |
| | Rhodococcus) | Oscillatoria, | Azobacter, |
| | Bacillus | Synechocystis, | Rhodobacter, |
| | Clostridium | Gloeocapsa, | Rhodopseudomonas, |
| | Beta Proteobacteria (e.g. | Hapalosiphon, | Methanosarcina, |
| | Achromobacter) | Stigonema, | Methanospirillum, |
| | | Microcoleus, | Methanobacterium |
| | | Aphanothece | |
| Microspora: | Gammaproteobacteria | Anabaena, | Klebsiella, |
| Microspora | (e.g. Escherichia, | Nostoc, | Bacillus, |
| | Pseudomonas) | Spirulina, | Escherichia, |
| | Actinobacteria (e.g. | Synechococcus, | Paenibacillus, |
| | Rhodococcus) | Oscillatoria, | Azobacter, |
| | Bacillus | Synechocystis, | Rhodobacter, |
| | Clostridium | Gloeocapsa, | Rhodopseudomonas, |
| | Beta Proteobacteria (e.g. | Hapalosiphon, | Methanosarcina, |
| | Achromobacter) | Stigonema, | Methanospirillum, |
| | | Microcoleus, | Methanobacterium |
| | | Aphanothece | |
| Xanthophyta: | Gammaproteobacteria | Anabaena, | Klebsiella, |
| Tribonemu, | (e.g. Escherichia, | Nostoc, | Bacillus, |
| | Pseudomonas) | Spirulina, | Escherichia, |
| | Actinobacteria (e.g. | Synechococcus, | Paenibacillus, |
| | Rhodococcus) | Oscillatoria, | Azobacter, |
| | Bacillus | Synechocystis, | Rhodobacter, |
| | Clostridium | Gloeocapsa, | Rhodopseudomonas, |
| | Beta Proteobacteria (e.g. | Hapalosiphon, | Methanosarcina, |
| | Achromobacter) | Stigonema, | Methanospirillum, |
| | | Microcoleus, | Methanobacterium |
| | | Aphanothece | |
| Rhodophyta: | Gammaproteobacteria | Anabaena, | Klebsiella, |
| Compsopogonopsis, | (e.g. Escherichia, | Nostoc, | Bacillus, |
| Audoouinella | Pseudomonas) | Spirulina, | Escherichia, |
| | Actinobacteria (e.g. | Synechococcus, | Paenibacillus, |
| | Rhodococcus) | Oscillatoria, | Azobacter, |
| | Bacillus | Synechocystis, | Rhodobacter, |
| | Clostridium | Gloeocapsa, | Rhodopseudomonas, |
| | Beta Proteobacteria (e.g. | Hapalosiphon, | Methanosarcina, |
| | Achromobacter) | Stigonema, | Methanospirillum, |
| | | Microcoleus, | Methanobacterium |
| | | Aphanothece | |

Particular aspects are directed to enhanced disruption and/or extraction of microorganisms to provide bio-products (e.g., biofuel, biocrude or bioenergy), wherein the inventive apparatus and methods differentially lyse particular microbial cells contained within a co-culture of at least one alga grown with at least one aerobic bacteria and at least one diazotroph. Particular aspects are directed to enhanced disruption and/or extraction of microorganisms to provide bio-products, wherein the aerobic bacteria of the co-culture are lysed before the algae, to provide for removal or bacterial lysis products prior to disruption and/or extraction of algal cells.

Certain aspects of the invention are directed to synergistic methods for enhanced disruption and/or extraction of microorganisms to provide bio-products (e.g., biofuel, biocrude or bioenergy, biogas, biodiesel, bioethanol, biogasoline, pharmaceuticals, nutraceuticals, food, vitamins, feedstock, dyes, colorants, sulfur, fertilizer, and bioplastic) depending on the microbial species. Certain methods of producing fuel grade oils and gases from algal biomass are known in the art (e.g., see, Dote, Yutaka, “Recovery of liquid fuel from hydrocarbon rich micro algae by thermo chemical liquefaction,” Fuel 73: Number 12. (1994); Ben-Zion Ginzburg, “Liquid Fuel (Oil) From Halophilic Algae: A renewable Source of Non-Polluting Energy, Renewable Energy,” 3:249-252, 1993; and Benemann, John R. and Oswald, William J., “Final report to the DOE: System and Economic Analysis of Micro algae Ponds for Conversion of CO₂ to Biomass.” DOE/PC/93204-T5, March 1996; each incorporated by reference herein for teachings of their respective methods).

According to preferred aspects, the disclosed synergitic osmotic shock methods are directed to enhanced disruption and/or extraction of microorganisms to provide bio-products (e.g., biofuel, biocrude or bioenergy, biogas, biodiesel, bioethanol, biogasoline, pharmaceuticals, nutraceuticals, food, vitamins, feedstock, dyes, colorants, sulfur, fertilizer, and bioplastic) from fungi. Background on prior art methods for obtaining products from fungi is found, for example in: Strobel G. et. al., “The production of myco-diesel hydrocarbons and their derivatives by the endophytic fungus Gliocladium roseum (NRRL 50072)”, Microbiology 154:3319-3328, 2008; and Takeno S. et. al., “Improvement of the Fatty Acid Composition of an Oil-Producing Filamentous Fungus, Mortierell alpine 1S-4, through RNA Interference with Δ12-Desaturase Gene Expression”, Applied and Environmental Microbiology 71:5124-5128, 2005, both incorporated herein by reference in their entirety for their respective above-described teachings.

### DEFINITIONS

“Microorganism”, as used herein, refers to organisms that are microscopic. Microorganisms include but are not limited to microbes, microbial cells, algae, bacteria, archaea, protist, yeast and fungi. Particular microorganisms useful for practice of the presently disclosed synergistic methods comprise both a cell wall and a cell membrane (e.g., algal cells).

“Algae”, as used herein, refers to a large and diverse group of simple, typically autotrophic organisms, ranging from unicellular to multicellular forms. The largest and most complex marine forms are called seaweeds. They are photosynthetic, like plants, and “simple” because they lack the many distinct organs found in land plants. By modern definitions, algae are eukaryotes and conduct photosynthesis within membrane-bound organelles called chloroplasts. Exemplary algae useful for practice of the presently disclosed synergistic methods comprise, but are not limited to those disclosed herein.

“Fungi”, as used herein, refers to organisms that are eukaryotic and typically having chitin cell walls but no chlorophyll or plastids. Fungi may be unicellular (e.g., yeast) or multicellular (e.g., molds). Exemplary fungi useful for practice of the presently disclosed synergistic methods comprise, but are not limited to those disclosed herein.

“Osmolarity” as used herein, refers to the measure of solute concentration, defined as the number of osmoles of solute per liter of solution (osmol/L).

“Osmolality” as used herein, refers to the measure of the osmoles of solute per kilogram of solvent (osmol/kg).

“Tonicity” as used herein, is a measure of the osmotic pressure of two solutions separated by a semipermeable membrane. Tonicity measures the ability of a solution to exert an osmotic pressure upon the membrane. It is commonly used when describing the response of cells immersed in an external solution. Like osmotic pressure, tonicity is influenced only by solutes that cannot cross the membrane, as only these exert an osmotic pressure. Solutes able to freely cross the membrane do not affect tonicity because they will always be in equal concentrations on both sides of the membrane. Thus tonicity, like osmolarity, also refers to the measure of solute concentration, but takes into account the total concentration of only non-penetrating solutes.

A “hypertonic” solution, as used herein, contains a greater concentration of impermeable solutes than the solution on the other side of the membrane. If a cell is placed in a hypertonic solution, there will be a net movement of water out of the cell until the concentration of impermeable solutes in the cell equals that of the hypertonic solution.

A “hypotonic” solution contains a smaller concentration of impermeable solutes than the solution on the other side of the membrane. If a cell is placed in a hypotonic solution, there will be a net movement of water into the cell until the concentration of impermeable solutes in the cell equals that of the hypotonic solution.

An “isotonic” solution contains an equal concentration of impermeable solutes as the solution on the other side of the membrane. If a cell is placed in an isotonic solution, there will be no net movement of water in or out of the cell because the concentration of impermeable solutes in the cell equals that of the external environment.

“Isoosmotic,” “hyperosmotic” and “hypoosmotic” versus “isotonic,” “hypertonic” and “hypotonic.” Osmolarity and tonicity are related, but different, and therefore, the terms ending in -osmotic (isoosmotic, hyperosmotic, hypoosmotic) are not synonymous with the terms ending in -tonic (isotonic, hypertonic, hypotonic). The terms are related in that they both compare the solute concentrations of two solutions separated by a membrane. The terms are different because osmolarity takes into account the total concentration of penetrating solutes and non-penetrating solutes, whereas tonicity takes into account the total concentration of only non-penetrating solutes. Penetrating solutes can diffuse through the cell membrane, causing momentary changes in cell volume as the solutes “pull” water molecules with them. Non-penetrating solutes cannot cross the cell membrane, and therefore osmosis of water must occur for the solutions to reach equilibrium. A solution can be both hyperosmotic and isotonic. For example, the intracellular fluid and extracellular fluid can be hyperosmotic, but isotonic if the total concentration of solutes in one compartment is different than the other, but where one of the ions can cross the membrane, drawing water with it and thus causing no net change in solution volume.

A “source medium” having a “source tonicity” as used herein refers to the tonicity of the normal growth and/or isolation medium (e.g., natural environment (saltwater, marine water, brackish water, fresh water, ground water, standard culture media for particular microorganisms, etc.) from which a microorganism is grown, derived and/or isolated, prior to imposition of the disclosed synergistic osmotic/tonicity shift/shock methods.

“Fresh water tonicity”, as used herein, includes but is not limited to fresh water, normal medium (e.g., natural environment (fresh water, ground water, normal culture medium, etc.), and a variety of laboratory culture media, wherein the tonicity of the solution includes approximately 0.0% to about 1.5% g/L total dissolved salt (TDS), or 0.1% to about 1.0% g/L TDS.

“Osmotic pressure”, as used herein, refers to the hydrostatic pressure produced by a solution in a space divided by a semi-permeable membrane due to a differential in the concentration of solute. For example, “osmotic pressure difference” can be the difference in the pressure exerted on the outside of the cell compared to the pressure exerted on the cellular membrane from inside the cell, which is due to interior of the cell having a different salt concentration than the cellular exterior. (e.g., see, Dinsdale and Walsby, “The Interrelations of Cell Turgot Pressure, Gas-vacuolation, and Buoyancy in a Blue-green Alga”, Journal of Experimental Botany, 23:561-570, 1972, which is incorporated by reference in its entirety).

“Salt” or “Salt water”, as used herein, refers to any salt or solution thereof that contains any type of salt, sugar, or solute molecule. For example, salt and salt water encompass any solute molecule or chemical that can alter the tonicity of a solution, including but not limited to NaCl, KCl, MgCl, NaSO₄, KSO₄, sugars (e.g., sucrose, glucose, glycerol, fructose and the like, carbonates, nitrates, etc.

“Total dissolved solids” or “total dissolved salt” as used herein, comprises inorganic salts and small amounts of organic matter that are dissolved in water. The principal constituents are usually the cations calcium, magnesium, sodium and potassium and the anions carbonate, bicarbonate, chloride, sulphate and in groundwater, nitrate. Concentrations of TDS in water vary owing to different mineral solubilities in different geological regions. The concentration of TDS in water in contact with granite, siliceous sand, well-leached soil or other relatively insoluble materials is usually below 30 mg/L. In areas of Precambrian rock, TDS concentrations in water are generally less than 65 mg/L. Levels are higher in regions of Palaeozoic and Mesozoic sedimentary rock, ranging from 195 to 1100 mg/L because of the presence of carbonates, chlorides, calcium, magnesium and sulphates.

According to particular aspects of the synergistic methods, the hypotonic osmotic pressure difference is equivalent to at least 0.25 g/L, at least 0.5 g/L, at least 0.75 g/L, at least 1 g/L, at least 1.25 g/L, at least 1.5 g/L, at least 1.75 g/L, at least 2 g/L, at least 2.25 g/L, at least 2.5 g/L, at least 2.75 g/L, at least 3 g/L, at least 3.25 g/L, at least 3.5 g/L, at least 3.75 g/L, at least 4 g/L, at least 5.25 g/L, at least 5.5 g/L, at least 5.75 g/L, at least 6 g/L, at least 6.25 g/L, at least 6.5 g/L, at least 6.75 g/L, at least 7 g/L, at least 7.25 g/L, at least 7.5 g/L, at least 7.75 g/L, at least 8 g/L, at least 8.25 g/L, at least 8.5 g/L, at least 8.75 g/L, at least 9 g/L, at least 9.25 g/L, at least 9.5 g/L, at least 9.75 g/L, at least 10 g/L, at least 10.25 g/L, at least 10.5 g/L, at least 10.75 g/L, and at least 11 g/L (% w/v or TDS).

According to particular aspects, the hypertonic and hypotonic solutions are made from any type of salt, or from combinations thereof. According to further aspects, the hypertonic solution is any concentration of salt above about 1.5 g/L TDS. According to preferred aspects of the synergistic methods, at least one hypertonic shock solution comprises salt in an amount between about 15 g/L and about 100 g/L TDS. According to preferred aspects of the synergistic methods, the hypertonic solution comprises salt in an amount between about 30 g/L and about 60 g/L TDS.

According to particular aspects of the synergistic methods, the hypotonic shock solution comprises salt in an amount between 0.0% to about 1.5% (g/L) total dissolved salt (TDS), or between about 0.1% to about 1.0% (g/L) TDS. Concentrations of TDS in drinking water, for example, are generally below 0.5 g/L (below 0.5% wt/v).

According to certain embodiments, the disclosed invention provides equipment and methods for synergistic osmotic shock pretreatment to enhance disruption and/or extraction of marine/saltwater/brackish water and freshwater microbial cells, using synergistic osmotic pressure shocking protocols. According to further embodiments, the synergistic osmotic shock methods are used prior to, or concomitantly with, cellular disruption and/or extraction methods, including but not limited to mechanical screw pressing, ultrasonic extraction, and solvent extraction, to enhance the extraction and isolation of bio-products (e.g., biofuel, biocrude or bioenergy, biogas, biodiesel, bioethanol, biogasoline, pharmaceuticals, nutraceuticals, food, vitamins, feedstock, dyes, colorants, sulfur, fertilizer, and bioplastic).

According to particular aspects, the disclosed methods are based, at least in part, on the scientific principle that the cell membrane and cell walls microbial cells can be subjected to osmotic pressure by incubating the cells in an environment of opposite or divergent tonicity (e.g., opposite in salt concentration) to their normal/natural habitat tonicity/osmolarity/osmolality. As appreciated in the art, for example, freshwater microbial cells can be osmotically shocked by incubation in saline solution, and marine microbial cells can be osmotically shocked by incubation in fresh water. According to particular aspects, when fresh water microbial cells are introduced into high salt concentration solution (hypertonic solution), the water inside the cells exits the cell and causes the cell wall to become fragile and instable and the cell walls loosen. According to further aspects, when marine cells are subjected to lower salt concentration solution than that inside the cells (hypotonic solution) such as fresh water, water enters the cells, causing the cells to swell. Sudden changes in osmotic pressure create osmotic shock with respect to the cell membrane and/or cell walls of microbial cells.

As appreciated in the art, there are interactions between the cell wall and the cell membrane of plants. For example, in plant cells suspended in a medium, the terms isotonic, hypotonic and hypertonic, which relate to solutes separated by a membrane, cannot strictly be used accurately because the pressure exerted by the cell wall significantly affects the osmotic equilibrium point. Additionally, as further appreciated in the art, a hypertonic environment forces water to leave a cell, such that in plant cells for example, in a process referred to as plasmolysis, the flexible cell membrane pulls away from the rigid cell wall, but remains joined, in pincushion fashion, to the cell wall at points called plasmodesmata, which become constricted and largely cease to function.

According to particular aspects of the present invention, the Applicant has discovered a unique synergistic method to productively destabilize, and thereby exploit, the apparent stabilizing interactions between the cellular membrane and the cell wall of plant cells. According to certain aspects, disruption of plant cells is facilitated by subjecting the plant cell wall to hypotonic shock after exposure of the cells to a hypertonic or isotonic medium, and imposing at least one final hypertonic shock. Without being bound by mechanism, according to particular aspects, the disruption of plant cells is facilitated by synergistically destabilizing the interaction between the cell wall and the cell membrane, such that the cell wall is more susceptible to shock-mediated weakening (e.g., hypotonic shock-mediated weakening).

In particular aspects the microbial cells are subjected to: a synergistic hypertonic/hypotonic/hypertonic tertiary shock; a synergistic hypotonic/hypertonic/hypertonic tertiary shock; or a synergistic hypotonic/hypertonic/hypotonic/hypertonic quaternary shock. Particular aspects further comprise disrupting and/or extracting of the synergistically shocked microbial cells, and isolating a cellular constituent or bioproduct (e.g., biofuel, biocrude, bioenergy, biogas, biodiesel, bioethanol, biogasoline, pharmaceuticals, nutraceuticals, food, vitamins, feedstock, dyes, colorants, sulfur, fertilizer, bioplastic) therefrom. In particular preferred aspects, synergistic facilitation of algae disruption and/or extraction is provided and in certain embodiments, at least one of lipid, oil, and triacylglycerol is isolated therefrom.

### Device for Enhanced Extraction

Particular aspects provide for a homogenizer or blades that allow for efficient mixing so salt can be added and mixed within the reactor without premixing the solution. This allows for a smaller reactor than would be necessary if saline solution was added. Particular aspects provide for a homogenizer having blades that can be easily changed. This allows for the blades to be either dull and low speed (e.g., for the purpose of mixing) or sharp (e.g., microbial cells that are large such as kelp (brown algae) and need to be cut in small pieces). As the blades are rotated they create high turbulence and thus the salt crystal can be mixed and dissolved with less energy consumption than ultrasonic machine. Thus, the equipment of the reactor associated with blades in this invention can be applied multi-functionally.

According to particular embodiments, the sieves are useful for filtering and separating the cellular debris and dust. According to further embodiments, sieve size can be from about 10 to about 200 mesh depending on cell sizes. In addition, multiple sieves of any size mesh can be used. The sieve is used to flush and release the saline solution while retaining the important cellular material. According to yet further embodiments, any one of the sieves can contain mesh that is made from any material including but not limited to metal, plastic, and fiber.

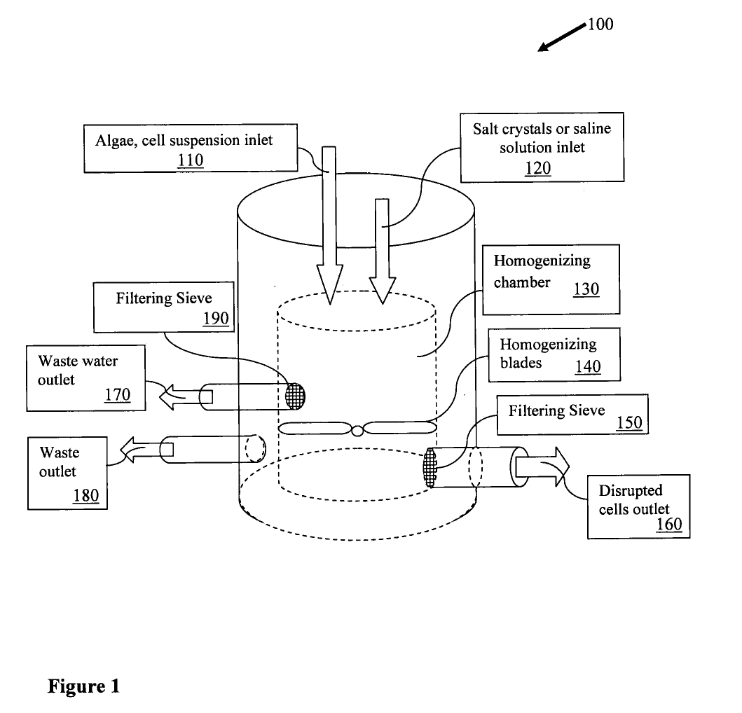

According to certain embodiments, the reactor contains both a liquid-solid inlet and outlet, and a liquid inlet and outlet. FIG. 1 shows, according to certain embodiments, the diagram of an exemplary reactor and where the inlet and the outlet are involved in changing the osmotic pressure of the solution within the reactor. The inlet and outlet maybe operatively connected to pumps that control the flow and/or addition of the liquids or the solutions as needed while operating the reactor.

According to certain embodiments, any part of the apparatus and/or reactor can be made of any material, including but not limited to, stainless steel, iron, glass, fiberglass, plastic, or metal. According to further embodiments, the reactor is operated in sequential batch protocol, or in single batch protocol. According to yet further embodiments, the reactor is operated in a continuous or semi-continuous protocol.

According to particular embodiments, the apparatus and methods for synergistic osmotic shock pretreatment to enhance the disruption and/or extraction of microbial cells to provide intracellular materials serves as a prelude or stage in harvesting and disruption and/or extraction of microbial or algal cells for various purposes. According to further embodiments, the apparatus and methods for synergistic osmotic shock pretreatment to enhance the disruption and/or extraction of microbial cells to provide intracellular materials serves as a prelude or stage in, for example, oil production from algae for biodiesel or biofuel. According to yet further embodiments, the apparatus and methods for synergistic osmotic shock pretreatment to enhance the disruption and/or extraction of microbial cells to provide intracellular materials is applied and operated as a continuous system and the entire equipment can create homogenization, synergistic osmotic pressure shock, and filtering continuously.

According to preferred embodiments, bio-products are more efficiently extracted from microbial samples (e.g., algae and fungi) after being subjected to the inventive pretreatment process using the apparatus and methods, as disclosed herein. For example, the inventive synergistic osmotic shock pretreatment process results in more efficient cellular disruption and/or solvent extraction, which in turn provides for higher yields (e.g., oil). Further, the synergistic osmotic shock methods result in the use of less solvent, time, and energy consumption for disrupting the microbial cells and extracting the intracellular materials.

In certain aspects, synergistic osmotic shock-mediated methods are useful for enhancing the disruption and/or extraction of mammalian cells.

Illustration of particular exemplary aspects of the invention is facilitated with reference to the following detailed description of the drawings.

FIG. 1 illustrates, according to particular exemplary aspects, a reactor 100 which consists of inlet 110 for algae cell or other microbial cell suspensions and inlet 120 for adding salt crystals or saline solution in case of fresh water algae or microorganisms or for adding fresh water in case of marine algae or microorganisms. Outlet 170 allows added saline solution or fresh water to drain from the homogenizing chamber through the sieve 190. Homogenizing blades 140 are fitted in the homogenizing chamber 130. The homogenizing blades 140 can be turned on at low speed at the steps to create mixing of salt and water for preparation of hypertonic solution. The outlet 160 from the homogenizing chamber 130 is fitted with an appropriate sieve 150. Unfiltered wastes exit through outlet 180.

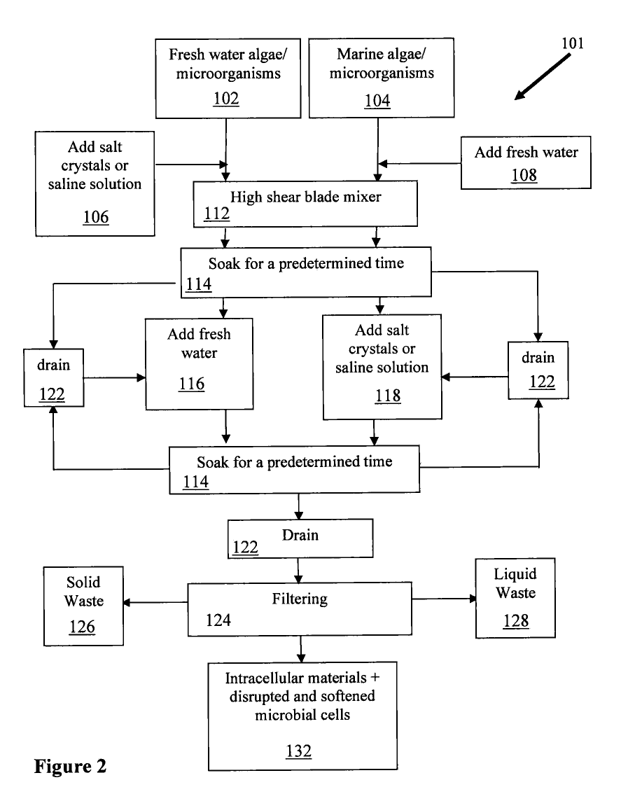

FIG. 2 summarizes, according to particular exemplary aspects, a flow chart 101 summarizing the process of the enhancement and extraction of intracellular materials from cells such as algae using the methods and apparatus of the invention. Fresh water algal/microorganismal 102 or marine algal/microorganismal 104 suspension enters the apparatus homogenizing chamber through inlet. Salt crystals or saline solution 106 is added to the fresh water algal/microorganismal 102. Fresh water 108 is added to the marine algal/microorganismal 104. Then the homogenizing blades are turned on at high speed 112 for at least 1 minute. The cells then are allowed to soak in the solution 114 for a predetermined time and then drained. Next in the process either fresh water is added 116 to the soaking cell suspension or salt crystals or saline solution is added 118 to the soaking cell suspension. The cells then may be drained 122 through outlet and saturate again with salt 118 or fresh water 116. This soaking 114 can be repeated as many times as required. The disrupted cells and their intracellular matters are filtered 124 which results in solid waste 126, liquid waste 128, and the intracellular material 132.

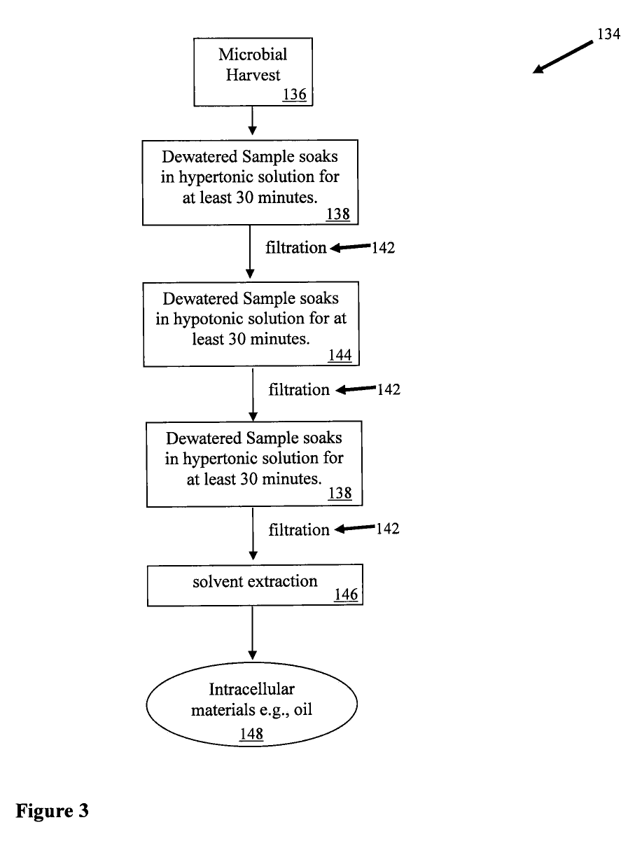

FIGS. 3-6 describe, according to particular exemplary aspects, methods for synergistically applying series of osmotic shocks for different types of algae or microorganisms depending on their natural aquatic habitat. FIG. 3 describes a method 134 wherein a fresh water microbial harvest 136 is subjected to (exposed to) alternating hypertonic and hypotonic shock steps. More specifically, the microbial harvest 136 is first exposed to a hypertonic solution 138 for 30 minutes. After this incubation, the mixture is filtered 142, and the sample treated with a hypotonic solution 144 for 30 minutes and then filtered 142 again. The filtered sample is treated (e.g., resuspended, or salt is added) with a hypertonic solution 138 for 30 minutes and then filtered 142 again. The filtered sample is subjected to solvent extraction 146 to obtain intracellular materials 148 (e.g., bio-product, bio-fuel, bio-ethanol).

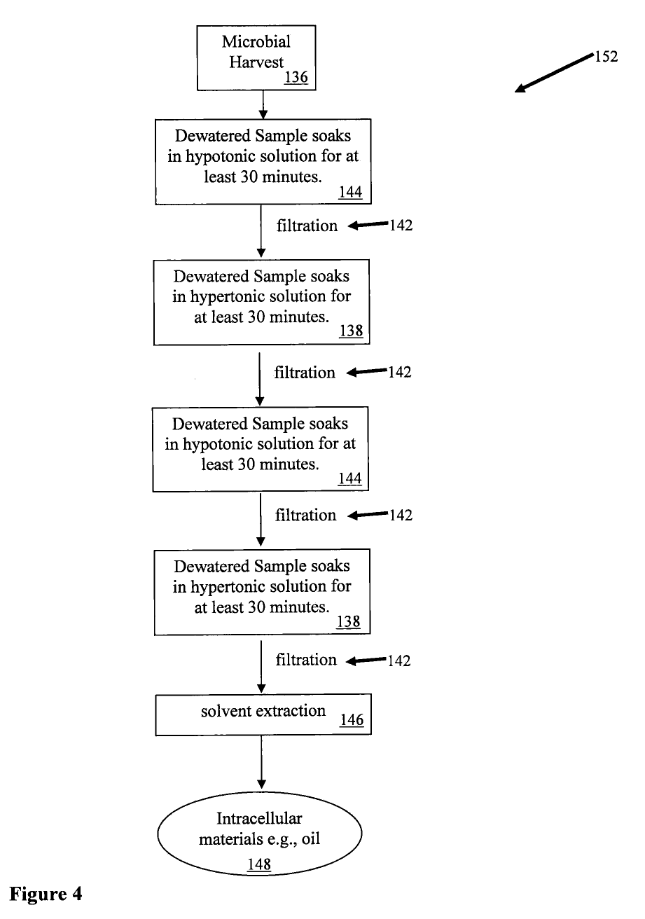

FIG. 4 describes a method 152 wherein a brackish/marine/saltwater microbial harvest 136 is synergistically subjected to alternating hypotonic and hypertonic shock steps. More specifically, the microbial harvest 136 is first exposed to a hypotonic solution 144 for 30 minutes. After this incubation, the mixture is filtered 142, and the sample is treated (e.g., resuspended, or salt is added) with a hypertonic solution 138 for 30 minutes and then filtered 142 again. The filtered sample is treated with a hypotonic solution 144 for 30 minutes and then filtered 142 again. The filtered sample is treated with a hypertonic solution 138 for 30 minutes and then filtered 142 again. Finally, the sample is subjected to solvent extraction 146 to obtain intracellular materials 148 (e.g., bio-product, bio-fuel, bio-ethanol).

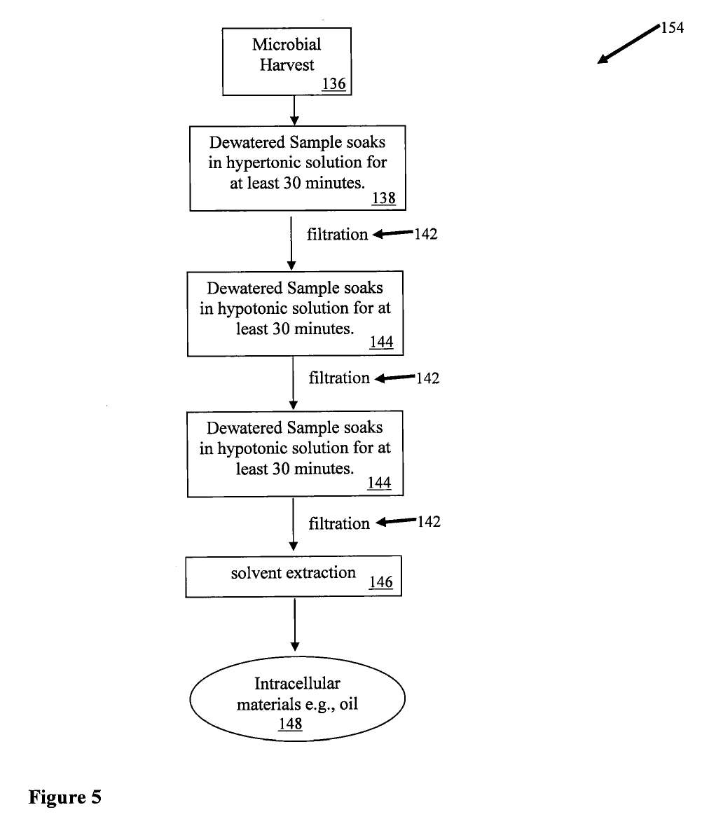

FIG. 5 describes a method 154 wherein a fresh water microbial harvest 136 is subjected to (exposed to) synergistic alternating hypertonic and hypotonic shock steps. More specifically, the microbial harvest 136 is first exposed to a hypertonic solution 138 for 30 minutes. After this incubation, the mixture is filtered 142, and the filtered sample is treated (e.g., resuspended) with a hypotonic solution 144 for 30 minutes and then filtered 142 again. The filtered sample is treated with another hypotonic solution 144 for 30 minutes and then filtered 142 again. Finally, the sample is subjected to solvent extraction 146 to obtain intracellular materials 148 (e.g., bio-product, bio-fuel, bio-ethanol).

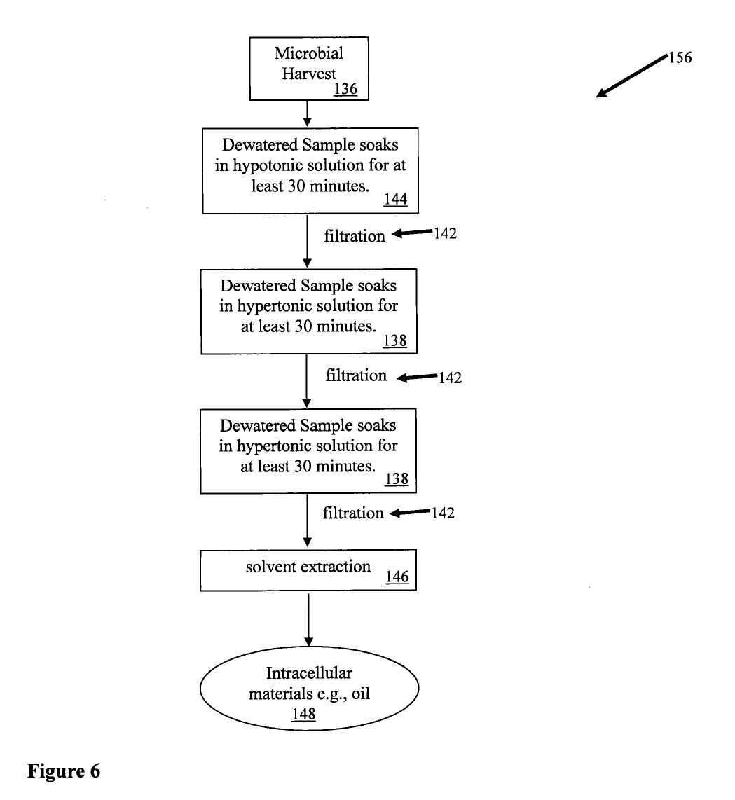

FIG. 6 describes a method 156 wherein a brackish/marine/saltwater microbial harvest 136 is synergistically subjected to alternating hypotonic and hypertonic. More specifically, the microbial harvest 136 is first exposed to a hypotonic solution 144 for 30 minutes. After this incubation, the mixture is filtered 142, and the filtered sample is treated with a hypertonic solution 138 for 30 minutes and then filtered 142 again. The filtered sample is treated with a hypertonic solution 138 for 30 minutes and then filtered 142 again. Finally, the sample is subjected to solvent extraction 146 to obtain intracellular materials 148 (e.g., bio-product, bio-fuel, bio-ethanol).

According to particular aspects, after the final shock step of the disclosed synergistic osmotic shock method the percent enhancement of disruption and/or extraction (relative to that of non-osmotic shock cells) is at least 15%, at least 20%, at least 25%, at least 30%, or at least 35%. Preferably the percent enhancement of disruption and/or extraction is at least 25%.

According to certain aspects, the period of incubation of microbial cells in the hypertonic solution is for a duration of between 1 minute and 9 hours. Preferably, the period of incubation of microbial cells in the hypertonic solution is between 5 minutes and 4 hours. Most preferably, the period of incubation of microbial cells in the hypertonic solution is between 10 minutes and 2 hours. In particular aspects, the period of incubation of microbial cells in the hypertonic solution is about 2 hours.

According to certain aspects, the period of incubation of microbial cells in the hypotonic solution is for a duration of between 1 minute and 9 hours.

Preferably, the period of incubation of microbial cells in the hypotonic solution is between 5 minutes and 4 hours. Most preferably, the period of incubation of microbial cells in the hypotonic solution is between 10 minutes and 2 hours. In particular aspects, the period of incubation of microbial cells in the hypotonic solution is less than or equal to 30 minutes, less than or equal to 15 minutes, less than or equal to 10 minutes, or less than or equal to 5 minutes.

### Alternate Exemplary Synergistic Osmotic Shock Embodiments:

Exemplary algal phyla and genera were subjected to four different methods of synergistic tonicity shifts as shown in Tables 3 and 4. On the left side of Table 3, an exemplary method of tonicity shifts used to treat fresh water algae is shown, wherein the tonicity utilized a hypertonic shift, a hypotonic shift, and a last hypotonic shift as shown. This was the method used for the experiment conducted in Example 4.

On the right side of Table 3, an exemplary method of synergistic tonicity shifts used to treat fresh water algae is shown, wherein the tonicity utilized a hypertonic shift, a hypotonic shift, and a last hypertonic shift as shown. This was the method used for the experiment conducted in Example 2.

On the left side of Table 4, an exemplary method of synergistic tonicity shifts used to treat salt/marine/brackish water algae is shown, wherein the tonicity protocol utilized a hypotonic shift, a hypertonic shift, and a last hypertonic shift is shown. This was the method used for the experiment conducted in Example 5.

On the right side of the table an exemplary method of synergistic tonicity shifts used to treat salt/marine/brackish water algae, wherein the tonicity protocol utilized a hypotonic shift, a hypertonic shift, a hypotonic shift, and a last hypertonic shift is shown. This was the method used for the experiment conducted in Example 3.

TABLE 3
Exemplary methods used for repetitively subjecting freshwater algae to a series of
synergistic osmotic shocks to enhance the disruption and/or extraction of cellular
or intracellular materials.

| Method 1 | | | | Method 2 | | |
|---|---|---|---|---|---|---|
| Steps | | | Algae phyla and | Steps | | |
| I | II | III | representative genera* | I | II | III |
| Hyper | Hypo | Hypo | Cyanophyta: | Hyper | Hypo | Hyper |
| | | | Anabaena, Nostoc, Spirulina, | | | |
| | | | Synechococcus, Oscillatoria, | | | |
| | | | Synechocystis, Chlorococcus | | | |
| Hyper | Hypo | Hypo | Chlorophyta: | Hyper | Hypo | Hyper |
| | | | Chlorella, Scenedesmus, | | | |
| | | | Chlamydomonas, Closterium, | | | |
| | | | Synedra, Pediastrum, Ankistrodesmus, | | | |
| | | | Planktosphaeria, Mougeotia, Spirogyra | | | |
| Hyper | Hypo | Hypo | Euglenophyta: | Hyper | Hypo | Hyper |
| | | | Euglena, | | | |
| Hyper | Hypo | Hypo | Bacillariophyta: | Hyper | Hypo | Hyper |
| | | | Navicula, Surirella | | | |
| Hyper | Hypo | Hypo | Microspora: | Hyper | Hypo | Hyper |
| | | | Microspora, | | | |
| Hyper | Hypo | Hypo | Xanthophyta: | Hyper | Hypo | Hyper |
| | | | Tribonemu, | | | |
| Hyper | Hypo | Hypo | Rhodophyta: | Hyper | Hypo | Hyper |
| | | | Compsopogonopsis, Audoouinella | | | |

*Genera has both fresh water and salt/marine/brackish water species

TABLE 4
Exemplary methods used for repetitively subjecting brackish water algae and
marine/salt water algae to a series of synergistic osmotic shocks to enhance the disruption and/or
extraction of cellular or intracellular materials.

| Method 1 | | | | Method 2 | | | |
|---|---|---|---|---|---|---|---|
| Steps | | | Algae phyla and representative | Steps | | | |
| I | II | III | genera* | I | II | III | IV |
| Hypo | Hyper | Hyper | Brackish Water Algae | Hypo | Hyper | Hypo | Hyper |
| | | | e.g. Cyanophyta: | | | | |
| | | | Spirulina, Lyngbya | | | | |
| Hypo | Hyper | Hyper | Bacillariophyta: | Hypo | Hyper | Hypo | Hyper |
| | | | diatom, Biddulphia, Staurophora | | | | |
| Hypo | Hyper | Hyper | Chlorophyta: | Hypo | Hyper | Hypo | Hyper |
| | | | Chlorella, Enteromorpha, | | | | |
| | | | Chaetomorpha, Rhizoclonium, | | | | |
| | | | Cladophorella, Klebsormidium, | | | | |
| | | | Botryococcus, Mougeotia, | | | | |
| Hypo | Hyper | Hyper | Marine/Salt water Algae | Hypo | Hyper | Hypo | Hyper |
| | | | e.g. Cyanophyta: | | | | |
| | | | Spirulina, Anabaena, Oscillatoria | | | | |
| Hypo | Hyper | Hyper | Chlorophyta: | Hypo | Hyper | Hypo | Hyper |
| | | | Chlorella, Scenedesmus, | | | | |
| | | | Chlamydomonas | | | | |
| Hypo | Hyper | Hyper | Bacillariophyta: | Hypo | Hyper | Hypo | Hyper |
| | | | diatom, Nitzschia, Navicula, | | | | |
| | | | Thalassiosira, Cocconeis, | | | | |
| | | | Chaetocero, Skeletonema | | | | |
| Hypo | Hyper | Hyper | Heterokontophyta: | Hypo | Hyper | Hypo | Hyper |
| | | | kelp, Macrocystis, Saccharina, | | | | |
| | | | Laminaria | | | | |
| Hypo | Hyper | Hyper | Phaeophyta: | Hypo | Hyper | Hypo | Hyper |
| | | | Nereocystis, Padina | | | | |
| Hypo | Hyper | Hyper | Rhodophyta | Hypo | Hyper | Hypo | Hyper |
| | | | Porphyra | | | | |

*Genera has both fresh water and salt/marine/brackish water species

### Example 1

### Materials and Methods

Growth conditions of algae. Either surface water (for fresh water species) or salt water (for brackish/marine/saltwater algae) was inoculated with at least one algal species. If needed (e.g., the algae is growing poorly) and/or to increase algae growth, a very low nutrient medium (as defined herein) was added to the surface water. The depth of the growth medium was kept constant at 40 cm by manually measuring the depth of the growth medium and adding growth medium sufficient to establish the proper depth, or the depth was adjusted automatically with a float ball. The temperature was maintained between 25-30° C. by adding cold water to the medium if the temperature is higher than 30° C. or heating by exchanging heat with waste steam if the temperature is lower than 25° C. Typically, the level of CO₂ was maintained within a range of about 1200 mg/L to about 1400 mg/L. Typically, the level of O₂ was maintained within a range of about 6 mg/L to about 50 mg/L. Typically, the level of nitrogen was maintained within a range of about 14 mg/L to about 18 mg/L. Typically, the pH is kept at a value between about pH 6.5 and pH 7.8 for optimal growth.

Co-culture growth conditions. Surface water was inoculated with at least one algal species, at least one aerobic bacteria, and at least one diazotrophic species in a range ratio of 10-1000:0.16-160:0.018-18, respectively (for specific ratios see examples disclosed herein). If needed (e.g., the algae is growing poorly) and/or to increase algae growth, a very low nutrient medium (as defined herein) was added to the surface water. The depth of the growth medium was kept constant at 40 cm by manually measuring the depth of the growth medium and adding growth medium sufficient to establish the proper depth, or the depth was adjusted automatically with a float ball. The temperature was maintained between 25-30° C. by adding cold water to the medium if the temperature is higher than 30° C. or heating by exchanging heat with waste steam if the temperature is lower than 25° C. Typically, the level of CO₂ was maintained within a range of about 1200 mg/L to about 1400 mg/L. Typically, the level of O₂ was maintained within a range of about 6 mg/L to about 50 mg/L. Typically, the level of nitrogen was maintained within a range of about 14 mg/L to about 18 mg/L, or preferably, an amount of nitrogen equivalent to less than about 5 mM sodium or potassium nitrate is used. More preferably, an amount of nitrogen equivalent to less than about 2 mM sodium or potassium nitrate is used. In certain embodiments about 0.5 mM (starved) to about 5 mM (deprived) of sodium or potassium nitrate was used. Typically, the pH is kept at a value between about pH 6.5 and pH 7.8 for optimal growth.

Very low nutrient media. Very low nutrient media was 100 g of KNO₃, 10 g of KH₂PO₄, 10 g of Na₂HPO₄, 1000 g of NaHCO₃, 1.5 g Fe-EDTA, 0.36 g of MnCl₂*4H₂O, 0.4 g of MgSO₄*7H₂O, 0.5 g of H₃BO₃, 0.3 g of ZnSO₄*7H₂O, 0.1 g of Na₂MoO₄*2H₂O, 0.016 g of CuSO₄*5H₂O, and 0.01 g Co(NO₃)₂*6H₂O per 1000 L of surface water.

Algae harvesting. Algae were harvested from the growth medium by skimming the top of the culture and/or collecting from the bottom or from the bulk of the vessel via pumping and filtering.

Algae harvesting of co-culture. Algae were harvested from the growth medium by skimming the top of the culture and/or collecting from the bottom or from the bulk of the vessel via pumping and filtering. The frequency and extent of algal harvesting was suitable to provide for sustained symbiotic co-culture of the algal, aerobic bacterial and diazotrophic organisms. Typically, only a fraction (e.g., 10 percent) of algae in the culture was harvested such that the algae remaining after harvest was sufficient to re-establish and/or sustain the symbiotic co-culture.

Hypertonic Solution. Exemplary hypertonic solutions are either 30 g/L or 60 g/L NaCl solution. Hypertonic solution can also, for example, be seawater, which is approximately 30 g/L NaCl solution. The composition of the particular salts in hypertonic solutions may, as will be appreciated in the art vary, while still providing suitable hypertonic solutions.

Hypotonic Solution. Exemplary hypotonic solutions include, but are not limited to, normal tap water. According to particular aspects of the synergistic methods, the hypotonic shock solution comprises salt in an amount between 0.0% to about 1.5% (g/L) total dissolved salt (TDS), or between about 0.1% to about 1.0% (g/L) (TDS).

Extraction methods. There are several extraction methods that can be used subsequent to the inventive apparatus and methods. These extraction methods include but are not limited to: supersonically enhanced solvent extraction, enzyme enhanced extraction, supercritical fluid extraction, and mechanical extraction (e.g., pressing).

Pressing. Harvested algae that have been subjected to the inventive apparatus and methods are subjected to physical mechanical pressing using various press configurations (e.g., screw, expeller, piston, etc) to extract intracellular materials.

Ultrasonic waves. Harvested algae that have been subjected to the inventive apparatus and methods are subjected to ultrasonic waves, which are used to create cavitation bubbles in the liquid medium. When these bubbles collapse near the cell walls, the resulting shock waves and liquid jets cause those cells walls to break and release their contents into a solvent.

Solvent extraction. Solvent extraction can follow common methods such as Bligh and Dyer (1959) or Kolarovic and Fournier (1986). In place of hexane, benzene, ether or other solvents may be utilized. Supercritical CO₂ can also be used as a solvent for the extraction of bio-product. In this method, CO₂ is liquefied under pressure and heated to the point that it becomes supercritical (having properties of both a liquid and a gas), allowing it to act as a solvent.

### Example 2

### Several Different Species of Fresh Water Algae were Subjected to a Synergistic Osmotic Shock Protocol by Alternating Hypertonic and Hypotonic Solutions

Example Overview. In this working Example 2, a synergistic osmotic shock method is described wherein fresh water algae/microorganisms were exposed to a synergistic hypertonic-hypotonic-hypertonic shock protocol. In this Example, the Applicant describes a method which results in enhanced extraction of bio-oil.

Specifically, fresh water algae, grown as described in Example 1, were harvested and subjected to the osmotic shock method similar to the method shown in FIG. 3 and using the apparatus described in FIG. 1. First, the homogenizing chamber was filled with hypertonic solution. Then, harvested fresh water algae were added to the vessel via the inlet. The homogenizing blades were operated for approximately one minute. Then the homogenized sample was incubated in the hypertonic solution in the homogenizing chamber for approximately 30 minutes. The hypertonic solution was drained through outlet and the chamber was filled with hypotonic solution. Next, the sample was incubated in the hypotonic solution in the homogenizing chamber for approximately 2 hours. The chamber was drained again then filled with hypertonic solution. Then, the sample is incubated in the hypertonic solution for approximately 2 hours. Last, the hypertonic solution was drained and the sample was collected and subjected to an hexane extraction, to obtain oil.

### Results

The results from this experiment are shown in Table 5. In this Example, Applicant observed that there was a significant increase in the percent enhancement of extraction after the second hypertonic solution incubation step (average percent enhanced extraction 33) when compared to the hypotonic solution incubation step (average percent enhanced extraction 16.8). Percent enhancement of extraction was measured by comparing the oil yield from the microbial material of each step of the synergistic osmotic shock method (hypertonic, hypotonic, and hypertonic) with the yield of oil from extraction of control microorganisms not subjected to any osmotic shock steps with hexane alone.

TABLE 5
Method of repetitively subjecting some exemplary genera of
freshwater algae to a synergistic series of osmotic shocks to enhance
the disruption and/or extraction of cellular or intracellular materials.

| | % enhancement of extraction* | | | | |
|---|---|---|---|---|---|
| Steps | Chlorella | Chlorococcus | Spirogyra | Spirulina | Anabaena |
| Hyper | 35 | 29 | 23 | 21 | 23 |
| Hypo | 18 | 20 | 20 | 12 | 14 |
| Hyper | 39 | 36 | 36 | 27 | 27 |

*% enhancement is calculated based on the difference between subjecting the sample to the inventive osmotic shock method followed by solvent extraction and subjecting the sample to solvent extraction alone.

The values are average value measured from multiple unidentified species of each genus.

### Example 3

### Several Different Species of Brackish/Marine/Salt Water Algae were Subjected to a Synergistic Osmotic Shock Protocol by Alternating Hypotonic and Hypertonic Solutions

Example Overview. In this working Example 3, an osmotic shock method is described wherein brackish/marine/saltwater algae/microorganisms were exposed to a synergistic hypotonic-hypertonic-hypotonic-hypertonic shock protocol.

Specifically, exemplary brackish/marine/salt water algae, grown as described in Example 1, were harvested and subjected to the osmotic shock method as shown in FIG. 4. First, the algae was harvested, as described in Example 1, and the sample was added to a beaker containing a hypotonic solution. The mixture was homogenized with a blender and incubated in the hypotonic solution for approximately 2 hours. Next, the suspension was passed through a filter and he sample was collected from the filter and suspended in a hypertonic solution for approximately 2 hours, and then passed through a filter again. Then, the dewatered sample was incubated in a hypotonic solution for 2 hours and passed through the filter again. The collected sample was suspended once more in a hypertonic solution and incubated in the hypertonic solution for 9 hours before being filtered. Last, the collected sample was subjected to hexane extraction, to obtain oil.

### Results

The results from this experiment are shown in Table 6. In this Example, Applicant observed that there was a significant increase in the percent enhancement of extraction after the second hypertonic solution incubation step (average percent enhanced extraction 35.5) when compared to the first hypertonic solution incubation step (average percent enhanced extraction 20.8). Percent enhancement of extraction was measured by comparing the oil yield from the microbial material of each step of the synergistic osmotic shock method (hypotonic, hypertonic, hypotonic, and hypertonic) with the yield of oil from extraction of control microorganisms not subjected to any osmotic shock steps with hexane alone.

TABLE 6
Method of repetitively subjecting some example genera of brackish/saltwater/marine
algae to a series of synergistic osmotic shocks to enhance the disruption and/or extraction of
cellular or intracellular materials.

| | % enhancement of extraction* | | | | | |
|---|---|---|---|---|---|---|
| | Brackish water algae | | | Marine/Salt water algae | | |
| Steps | Spirulina | Chlorella | Botryococcus | Oscillatoria | Chlorella | Scenedesmus |
| Hypo | 4 | 6 | 7 | 6 | 6 | 5 |
| Hyper | 16 | 22 | 19 | 14 | 28 | 26 |
| Hypo | 8 | 10 | 12 | 9 | 14 | 16 |
| Hyper | 26 | 37 | 38 | 26 | 42 | 44 |

*% enhancement is calculated based on the difference between subjecting the sample to the inventive osmotic shock method followed by solvent extraction and subjecting the sample to solvent extraction alone.

The values are average value measured from multiple unidentified species of each genus.

### Example 4

### Several Different Species of Fresh Water Algae were Subjected to a Synergistic Osmotic Shock Protocol by Alternating Hypertonic and Hypotonic Solutions

Example Overview. In this working Example 4, an osmotic shock method is described wherein fresh water algae/microorganisms were exposed to a synergistic hypertonic-hypotonic-hypotonic shock protocol.

Specifically, fresh water algae/microorganisms, grown as described in Example 1, were harvested and subjected to the osmotic shock method as shown in FIG. 5. First, the homogenizing chamber was filled with hypertonic solution. Next, the harvested fresh water algae were added to the vessel via the inlet and the homogenizing blades were operated for approximately one minute. Then, the homogenized sample was incubated in the hypertonic solution in the homogenizing chamber for approximately 2 hours. Next, the hypertonic solution was drained through the outlet and the chamber was filled with hypotonic solution. The sample was incubated in the hypotonic solution in the homogenizing chamber for approximately 2 hours. Next, the chamber was drained and filled with yet another hypotonic solution. The sample was incubated in the hypotonic solution for approximately 2 hours after that the solution was drained, the sample collected and subjected to hexane extraction, to obtain oil.

### Results

The results from this experiment are shown in Table 7. In this Example, Applicant discovered that there was a significant decrease in the recovery of oil extraction after the second hypotonic solution incubation step (average percent enhanced extraction 16.7) when compared to the initial hypertonic solution incubation step (average percent enhanced extraction 34.3). Percent enhancement of extraction was measured by comparing the oil yield from the microbial material of each step of the inventive osmotic shock method (hypertonic, hypotonic, and hypotonic) with the yield of oil from extraction of control microorganisms not subjected to any osmotic shock steps with hexane alone.

TABLE 7
Method of repetitively subjecting some exemplary genera of
freshwater algae to a series of synergistic osmotic shocks to
enhance the disruption and/or extraction of cellular or
intracellular materials.

| | % enhancement of extraction* | | |
|---|---|---|---|
| Steps | Chlorella | Scenedesmus | Oscillatoria |
| Hyper | 36 | 38 | 29 |
| Hypo | 29 | 33 | 22 |
| Hypo | 16 | 19 | 15 |

*% enhancement is calculated based on the difference between subjecting the sample to the inventive osmotic shock method followed by solvent extraction and subjecting the sample to solvent extraction alone.

The values are average value measured from multiple unidentified species of each genus.

### Example 5

### Several Different Species of Brackish/Marine/Salt Water Algae were Subjected to a Synergistic Osmotic Shock Protocol by Alternating Hypotonic and Hypertonic Solutions

Example Overview. In this working Example 5, an osmotic shock method is described wherein brackish/marine/salt water algae/microorganisms were exposed to a synergistic hypotonic-hypertonic-hypertonic shock protocol.

Specifically, brackish/marine/salt water algae, grown as described in Example 1, were harvested and subjected to the osmotic shock method as shown in FIG. 6. For a large sample, an apparatus such as that illustrated in FIG. 1 was used. First, the homogenizing chamber was filled with hypotonic solution. Then, harvested brackish/marine/salt water algae were added to the vessel via the inlet and the homogenizing blades were operated for approximately one minute. Next, the homogenized sample was incubated in the hypertonic solution in the homogenizing chamber for approximately 2 hours. Then, the hypotonic solution was drained through outlet and the chamber was filled with hypertonic solution instead. Next, the sample was incubated in the hypertonic solution in the homogenizing chamber for approximately 2 hours. The chamber was drained and filled subsequently with a hypertonic solution. The sample was incubated in the hypertonic solution for approximately 2 hours. Last, the sample was collected and subjected hexane extraction, to obtain oil.

### Results

The results from this experiment are shown in Table 8. In this Example, Applicant discovered that there was a significant increase in the percent enhancement of extraction after the second hypertonic solution incubation step (average percent enhanced extraction 31.6) when compared to the first hypertonic solution incubation step (average percent enhanced extraction 19.8). Percent enhancement of extraction was measured by comparing the oil yield from the microbial material of each step of the inventive osmotic shock method (hypotonic, hypertonic, and hypertonic) with the yield of oil from extraction of control microorganisms not subjected to any osmotic shock steps with hexane alone.

TABLE 8
Method of repetitively subjecting some exemplary genera of brackish/saltwater/marine
algae to a series of synergistic osmotic shocksto enhance the disruption and/or extraction of
cellular or intracellular materials.

| | % enhancement of extraction* | | | | | |
|---|---|---|---|---|---|---|
| | Brackish water algae | | | Marine/Salt water algae | | |
| Steps | Spirulina | Chlorella | Botryococcus | Oscillatoria | Chlorella | Scenedesmus |
| Hypo | 4 | 6 | 6 | 3 | 5 | 6 |
| Hyper | 15 | 19 | 21 | 14 | 24 | 26 |
| Hyper | 23 | 33 | 36 | 25 | 36 | 37 |

*% enhancement is calculated based on the difference between subjecting the sample to the inventive osmotic shock method followed by solvent extraction and subjecting the sample to solvent extraction alone.

The values are average value measured from multiple unidentified species of each genus.

### Example 6

### Several Different Species of Fungi are Subjected to a Synergistic Osmotic Shock Protocol by Alternating Hypertonic and Hypotonic Solutions

Example Overview. In this working Example 6, an osmotic shock method is described wherein different species of fungi are exposed to a synergistic hypertonic-hypotonic-hypertonic shock protocol.

Specifically, several exemplary strains of fungi are harvested and subjected to the osmotic shock method as shown in FIG. 3. First, for a small amount of harvested sample, the sample is added to a beaker containing a hypertonic solution. Next, the mixture is homogenized with a blender and incubated in the hypertonic solution for approximately 2 hours. Then, the cellular suspension is passed through a filter. Next, the sample is collected from the filter and suspended in a hypotonic solution for approximately 2 hours before passing through a filter. Then, the dewatered sample is incubated in a hypertonic solution for 2 hours and passed through another filter. Last, the collected sample is subjected to an extraction process, such as solvent extraction using hexane, to obtain oil. Percent enhancement of extraction is measured by comparing the oil yield from the microbial material of each step of the inventive osmotic shock method (hypertonic, hypotonic, and hypertonic) with the yield of oil from extraction of control microorganisms not subjected to any osmotic shock steps with hexane alone.

### Example 7

### Comparison of the Oil Yield Obtained from Chlorella vulgaris, Chlorella Sp. D101 Using a Synergistic Osmotic Shock Protocol, Wherein the Incubation Time with the Hypotonic Solution was Increased Incrementally

Example Overview. In this working Example 7, synergistic osmotic shock methods were compared, wherein Chlorella vulgaris, Chlorella sp. D101 is subjected to increasing incubation times of the hypotonic shock step of the protocol.

Experimental Design. Parameters were kept similar to Example 4, but during the first step, the hypotonic step, the incubation time varied from 5 to 480 minutes. More specifically, during the hypotonic step samples were taken at the 5 minute, 15 minute, 30 minute, 60 minute, 120 minute, 240 minute, and 480 minute time points and each of the time point samples was subsequently split into three samples. One of the three samples from each time point was directly extracted with hexane, to determine the percent enhanced extraction of oil when compared to hexane extraction alone of control microorganismal material (no osmotic shock). The remaining two samples from each time point were incubated in a hypertonic solution for approximately two hours. After hypertonic solution incubation one of the two remaining samples was subject to hexane extraction to obtain the oil sample. The last remaining sample from each time point was incubated with another hypertonic solution for approximately two hours. After this last hypertonic incubation period, the remaining time point samples were collected and extracted with hexane to obtain oil. Percent enhancement of extraction was measured by comparing the oil yield from the microbial material of each step of the inventive osmotic shock method (hypertonic, hypotonic, and hypertonic) with the yield of oil from extraction of control microorganisms not subjected to any osmotic shock steps with hexane alone

### Results

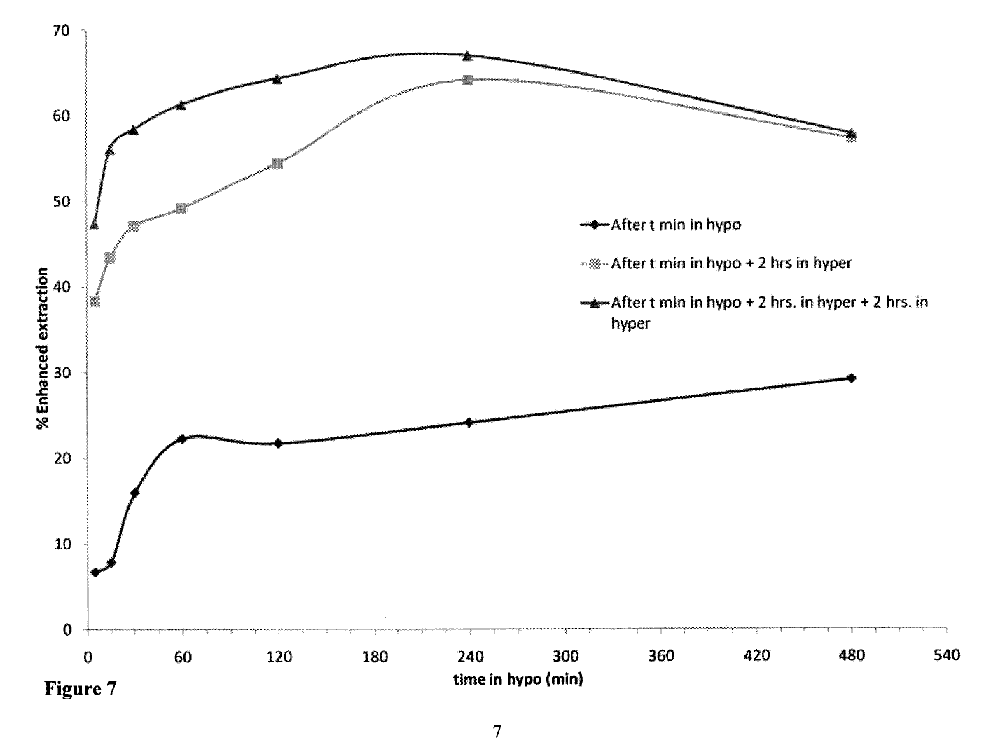

The results from this experiment are shown in FIG. 7. The bottom line in the figure is a curve representing each time point (the 5 minute, 15 minute, 30 minute, 60 minute, 120 minute, 240 minute, and 480 minute time points) that was taken during the first hypotonic incubation period and then immediately subjected to oil extraction. The middle line in the figure is a curve representing the material from each hypotonic time point taken and subjected to the second incubation (the first hypertonic incubation), which was then extracted for oil. The top line in the figure is a curve representing the material from each time point taken, subjected to the second incubation (the first hypertonic incubation), and a subsequent third incubation (the second hypertonic incubation) which was then extracted for oil. The curve shows the percent enhanced oil extraction, which is a percent increase of the amount of oil extracted from the microbial material with the inventive osmotic shock procedure and subsequently extracting with hexane, as compared to hexane extraction alone (no osmotic shock) of control microorganisms not subjected to any osmotic shock steps. Applicant discovered, based on the enhanced yield data as shown in FIG. 7, that surprisingly the hypotonic incubation step reaches peak efficiency within an hour, and moreover is significantly efficacious over much shorter periods of time. In addition, the results indicate the importance, as disclosed herein, of the subsequent synergistic hypertonic incubation steps. According to the second hypertonic step, the inventive osmotic shock methods resulted in enhanced extraction of oil of greater than 45% occurring by only the first 5 minutes of incubation with the hypotonic solution.

According to additional aspects, therefore, performing the inventive synergistic osmotic shock protocols using a relatively short hypotonic incubation time can yet provide for enhanced overall recovery of a bioproducts (e.g., oil), but in significantly less time, and further, in cases where small metabolites are sought (or where bioproducts that have a tendency to leach out from the cells during the osmotic shock protocol are sought), shock protocols using a relatively short hypotonic incubation time can minimize product loss during the shock protocols, and thus further enhance recovery in addition to the benefit of the synergistic shock.

Further embodiments comprise shortening at least one of the at least two hypertonic incubation periods for similar reasons and benefit.

According to yet further aspects, decreasing the temperature during the osmotic shock protocol, during either hypotonic or hypertonic, or both, steps further decreases the amount of bioproduct loss (e.g., by leaching) during the synergistic shock protocols. Preferably, the hypotonic shock step is performed with at least one of a decreased incubation time, and a decreased incubation temperature. For example, a temperature below ambient temperature can be used during the at least one hypotonic shock or during at least one of the at least two hypertonic shock steps, or a temperature between about 0° C. and about 25° C. can be used during the at least one hypotonic shock or during at least one of the at least two hypertonic shock steps.

### Example 8

### Analysis of Enhanced Disruption and/or Extraction of Bio-Products from Symbiotic Co-Cultures of Chlorella vulgaris, Chlorella sp. D101, Bacillus sp. D320, Rhodobacter sphaeroides, Rhodobacter sp. D788, and Spirulina maxima, Spirulina sp. D11

Example Overview. In this working Example 8, a continuous, symbiotic sustainable co-culture was harvested and subjected to a synergistic osmotic shock protocol to facilitate disruption of the algae and extraction of oil therefrom.

Specifically, a production culture for continuous and symbiotic algal growth was established by inoculating surface water with algal species Chlorella vulgaris, Chlorella sp. D101, two aerobic bacterial species Rhodobacter sphaeroides, Rhodobacter sp. D788 and Bacillus sp D320, and diazotrophic bacterial species Spirulina maxima, Spirulina sp. D11. The production culture vessel was a rectangular open plastic container having the dimensions of 1.25×2.75 m². Growth medium (surface water) was added via batch flow to the vessel to a depth of 40 cm and circulated by using a pump. Throughout the experiment, the depth of the growth medium was kept constant at 40 cm by manual addition or automatically with a float ball, as described under Example 1. Natural sunlight was used and was continuously cycled in alternating periods of approximately 12 hours of light and 12 hours of darkness. The temperature was maintained between 25-30° C. by cold water circulation or heating by exchanging heat with waste steam as described under Example 1.

Periodically, throughout the growth cycle Chlorella vulgaris, Chlorella sp. D101 was harvested from the growth medium by skimming the top of the culture and/or collecting from the bottom and/or from the bulk of the vessel via pumping and/or filtering. The frequency and extent of algal harvesting was suitable to provide for sustained symbiotic co-culture of the algal, aerobic bacterial and diazotrophic organisms. Typically, only a fraction (e.g., 10%, 25%, 30%, 35%, 40%, 45%, or 50%) of algae in the culture was harvested such that the algae remaining after harvest is sufficient to re-establish and/or sustain the symbiotic co-culture.

The harvested Chlorella vulgaris, Chlorella sp. D101 was then subject the osmotic shock method as described in Example 2. The finally collected sample was subjected to an extraction process, such as solvent extraction using hexane, to obtain oil. Percent enhancement of extraction was measured by comparing the oil yield from the microbial material subjected to the synergistic osmotic shock method (hypertonic, hypotonic, and hypertonic) with the yield of oil from extraction with hexane alone of control microorganisms not subjected to the synergistic osmotic shock method.

The synergistic methods resulted in an enhanced oil yield of at least 20-25%.

### Example 9

### Analysis of Enhanced Disruption and/or Extraction of Bio-Products from Symbiotic Co-Cultures of Chlorella vulgaris, Chlorella sp. D101, Bacillus sp D320, Rhodobacter sphaeroides, Rhodobacter sp. D788, Methanobacteria sp D422, and Spirulina maxima, Spirulina sp. D11

Example Overview. In this working Example 9, a continuous, symbiotic sustainable co-culture, was harvested and subjected to a synergistic osmotic shock protocol to facilitate disruption of the algae and extraction of oil therefrom.

Specifically, a production culture for continuous and symbiotic algal growth was established by inoculating surface water with algal species Chlorella vulgaris, Chlorella sp. D101, two aerobic bacterial species Rhodobacter sphaeroides, Rhodobacter sp. D788 and Bacillus sp D320, and two diazotrophic bacterial species Spirulina maxima, Spirulina sp. D11 and Methanobacteria sp D422. All parameters in this Example were identical to those disclosed in Example 8, except that an additional diazotroph, Methanobacteria sp D422, was added to the co-culture. Typically, in this Example, the relative amounts of the algal including cyanobacteria, aerobic bacterial and diazotrophic organisms (bacteria and archaea excluding cyanobacteria) were maintained in a ratio or proportion of about 100:1.6:0.18, respectively.

The harvested Chlorella vulgaris, Chlorella sp. D101 was then subject the osmotic shock method as described in Example 2. The finally collected sample was subjected to an extraction process, such as solvent extraction using hexane, to obtain oil. Percent enhancement of extraction was measured by comparing the oil yield from the algal material subjected to the synergistic osmotic shock method (hypertonic, hypotonic, and hypertonic) with the yield of oil from extraction (hexane) of control algal material not subjected to the synergistic osmotic shock method.

The synergistic methods resulted in an enhanced oil yield of at least 20-25%.

### Example 10

### Analysis of Enhanced Disruption and/or Extraction of Bio-Products from Symbiotic Co-Cultures of Scenedesmus obliquus, Scenedesmus sp. D202, Bacillus sp D320, Rhodobacter sphaeroides, Rhodobacter sp. D788, Methanobacteria sp D422, and Spirulina maxima, Spirulina sp. D11

Example Overview. In this working Example 10, a continuous, symbiotic sustainable co-culture was harvested and subjected to a synergistic osmotic shock protocol to facilitate disruption of the algae and extraction of oil therefrom.

Specifically, a production culture vessel for continuous and symbiotic growth was established by inoculating with algal species Scenedesmus obliquus, Scenedesmus sp. D202, two aerobic bacterial species Bacillus sp D320 and Rhodobacter sphaeroides, Rhodobacter sp. D788, and two diazotrophic bacterial species Methanobacteria sp D422 and Spirulina maxima, Spirulina sp. D11. Typically, in this Example, the relative amounts of the algal including cyanobacteria, aerobic bacterial and diazotrophic organisms (bacteria and archaea excluding cyanobacteria) were maintained in a ratio or proportion of about 100:1.6:0.18, respectively. The production culture vessel was an open raceway concrete vessel container having the dimensions of 6×40 m². Growth medium (surface water) was added via inlet valve to the vessel to a depth of 1 m and circulated via paddle wheel. Throughout the experiment, the depth of the growth medium was kept constant at 1 m by manual addition or automatically with a float ball, as described under Example 1. Natural sunlight was used and was continuously cycled in alternating periods of approximately 12 hours of light and 12 hours of darkness. The temperature was maintained between 25-30° C. by cold water circulation or heating by exchanging heat with waste steam as described under Example 1. The remaining parameters were identical to those disclosed in Example 8.

Periodically, throughout the growth cycle Scenedesmus obliquus, Scenedesmus sp. D202 was harvested from the growth medium by skimming the top of the culture and/or collecting from the bottom or from the bulk of the vessel via pumping and filtering. The frequency and extent of algal harvesting was suitable to provide for sustained symbiotic co-culture of the algal, aerobic bacterial and diazotrophic organisms. Typically, only a fraction (e.g., 10%, 15%, 20%, 25%, 30%, 35%, 40%, 45%, or 50%) of algae in the culture was harvested such that the algae remaining after harvest is sufficient to maintain (e.g., re-establish and/or sustain) the symbiotic co-culture.

The harvested Scenedesmus obliquus, Scenedesmus sp. D202 was then subject the osmotic shock method as described in Example 3. The finally collected sample was subjected to an extraction process, such as solvent extraction using hexane, to obtain oil.

Percent enhancement of extraction was measured by comparing the oil yield from the microbial material subjected to the synergistic osmotic shock method (hypotonic, hypertonic, hypotonic, and hypertonic) with the yield of oil from extraction (hexane) of control microorganisms not subjected to the synergistic osmotic shock method.

The synergistic methods resulted in an enhanced oil yield of at least 20-25%.

### Example 11

### Analysis of Enhanced Disruption and/or Extraction of Bio-Products from Symbiotic Co-Cultures of Euglena gracilis, Euglena sp. D405, Bacillus sp D320, Rhodobacter sphaeroides, Rhodobacter sp. D788, Methanobacteria sp D422, and Spirulina maxima, Spirulina sp. D11

Example Overview. In this working Example 11, a continuous, symbiotic sustainable co-culture was harvested and subjected to a synergistic osmotic shock protocol to facilitate disruption of the algae and extraction of oil therefrom.

Specifically, a production culture vessel for continuous and symbiotic growth was established by inoculating with algal species Euglena gracilis, Euglena sp. D405, the two aerobic bacterial species Bacillus sp D320 and Rhodobacter sphaeroides, Rhodobacter sp. D788, and two diazotrophic bacterial species Methanobacteria sp D422 and Spirulina maxima, Spirulina sp. D11. Typically, in this Example, the relative amounts of the algal including cyanobacteria, aerobic bacterial and diazotrophic organisms (bacteria and archaea excluding cyanobacteria) were maintained in a ratio or proportion of about 100:1.6:0.18, respectively. The production culture vessel comprised an open concrete vessel container having the dimensions of 25×1500 m². Growth medium was added from a tap through sand filter to the vessel to a depth of 1.2 m and circulated using a paddle wheel-type device. Throughout the experiment, the depth of the growth medium was kept constant at 1.2 m by manual addition or automatically with a float ball, as described under Example 1. Natural sunlight was used and was continuously cycled in alternating periods of approximately 12 hours of light and 12 hours of darkness. The temperature was maintained between 25-30° C. by cold water circulation or heating by exchanging heat with waste steam as described under Example 1. The remaining parameters were identical to those disclosed in Example 8.

Periodically, throughout the growth cycle Euglena gracilis, Euglena sp. D405 was harvested from the growth medium by skimming the top of the culture and/or collecting from the bottom or from the bulk of the vessel via pumping and filtering. The frequency and extent of algal harvesting was suitable to provide for sustained symbiotic co-culture of the algal, aerobic bacterial and diazotrophic organisms. Typically, only a fraction (e.g., 10%, 15%, 20%, 25%, 30%, 35%, 40%, 45%, or 50%)) of algae in the culture was harvested such that the algae remaining after harvest is sufficient to re-establish and/or sustain the symbiotic co-culture.

The harvested Euglena gracilis, Euglena sp. D405 was then subject the osmotic shock method as described in Example 2. The finally collected sample was subjected to an extraction process, such as solvent extraction using hexane, to obtain oil.

Percent enhancement of extraction was measured by comparing the oil yield from the microbial material subjected to the synergistic osmotic shock method (hypertonic, hypotonic, and hypertonic) with the yield of oil from extraction (hexane) of control microorganisms not subjected to the synergistic osmotic shock method.

The synergistic methods resulted in an enhanced oil yield of at least 20-25%.

1. A method for enhancing disruption or extraction of microbial cells, comprising:
obtaining microbial cells, the microbial cells having a cell membrane and having been grown in a source medium having a source tonicity with respect to said membrane;
suspending the microbial cells, for a primary shock time period, in an initial aqueous suspension medium having a primary tonicity different from the source tonicity to provide primary shocked microbial cells;
suspending the primary shocked microbial cells, for a secondary shock time period, in an secondary aqueous suspension medium having a secondary tonicity different from the primary tonicity to provide secondary shocked microbial cells;
suspending the secondary shocked microbial cells, for a tertiary shock time period, in an tertiary aqueous suspension medium having a tertiary tonicity that is the same or different from the secondary tonicity to provide tertiary shocked microbial cells; and
subjecting the tertiary shocked microbial cells to a suitable disruption method, wherein the tonicity shocking comprises a synergistic combination of at least one hypotonic shock and at least two hypertonic shocks, and wherein the last tonicity shock is a hypertonic shock, wherein enhancing at least one of disruption and extraction of microbial cells is afforded.

2. The method of claim 1, wherein the source medium is fresh water having a fresh water tonicity, wherein the primary tonicity is hypertonic, and wherein the microbial cells are subjected to a hypertonic/hypotonic/hypertonic tertiary shock.

3. The method of claim 1, wherein the source medium is saltwater, brackish water, or marine water, having a salt, brackish or marine water tonicity, respectively, wherein the primary tonicity is hypotonic, and wherein the microbial cells are subjected to a hypotonic/hypertonic/hypertonic tertiary shock.

4. The method of claim 1, further comprising, after the tertiary shock time period and prior to subjecting to a suitable disruption period, suspending the tertiary shocked microbial cells, for a quaternary shock time period, in an quaternary aqueous suspension medium having a quaternary tonicity that is different from the tertiary tonicity to provide quaternary shocked microbial cells; and subjecting the quaternary shocked microbial cells to a suitable disruption and/or method.

5. The method of claim 4, wherein the source medium is saltwater, brackish water, or marine water, having a salt, brackish or marine water tonicity, respectively, wherein the primary tonicity is hypotonic, and wherein the microbial cells are subjected to a hypotonic/hypertonic/hypotonic/hypertonic quaternary shock.

6. The method of any one of claims 1 and 4, wherein the at least one hypotonic shock comprises a hypotonic shock equivalent to at least 5 g/L total dissolved salt (TDS), and wherein at least one of the at least two hypertonic shocks comprises a hypertonic shock equivalent to at least 15 g/L TDS.

7. The method of claim 6, wherein the at least one hypotonic shock comprises a hypotonic shock equivalent to a value in the range of from about 8 g/L to about 12 g/L total dissolved salt (TDS), and wherein at least one of the at least two hypertonic shocks comprises a hypertonic shock in the range of from about 15 g/L to about 60 g/L total dissolved salt (TDS).

8. The method of claim 6, wherein the at least one hypotonic shock comprises a hypotonic shock equivalent to a value in the range of from about 9 g/L to about 10 g/L total dissolved salt (TDS), and wherein at least one of the at least two hypertonic shocks comprises a hypertonic shock in the range of from about 20 g/L to about 35 g/L total dissolved salt (TDS).

9. The method of claim 1, wherein at least one of suspending the microbial cells, suspending the primary shocked microbial cells, and suspending the secondary shocked microbial cells comprises addition of saline to the suspension medium.

10. The method of claim 1, wherein at least one of suspending the microbial cells, suspending the primary shocked microbial cells, and suspending the secondary shocked microbial cells comprises changing the suspension medium.

11. The method of claim 1, wherein at least one of suspending the microbial cells, suspending the primary shocked microbial cells, and suspending the secondary shocked microbial cells comprises diluting the suspension medium.

12. The method of claim 4, wherein at least one of suspending the microbial cells, suspending the primary shocked microbial cells, suspending the secondary shocked microbial cells, and suspending the tertiary shocked microbial cells, comprises addition of saline to the suspension medium.

13. The method of claim 4, wherein at least one of suspending the microbial cells, suspending the primary shocked microbial cells, suspending the secondary shocked microbial cells, and suspending the tertiary shocked microbial cells, comprises changing the suspension medium.

14. The method of claim 4, wherein at least one of suspending the microbial cells, suspending the primary shocked microbial cells, suspending the secondary shocked microbial cells, and suspending the tertiary shocked microbial cells, comprises diluting the suspension medium.

15. The method of any one of claims 1 and 4, wherein the primary shock time period, the secondary shock time period, the tertiary shock time period, and the quaternary shock time period are equal or substantially equal in duration.

16. The method of claim 15, wherein the duration is within a duration range of about 0.5 hr to about 4 hr.

17. The method of any one of claims 1 and 4, wherein the duration of at least two of the primary shock time period, the secondary shock time period, the tertiary shock time period, and the quaternary shock time period differ.

18. The method of any one of claims 1 and 4, wherein the duration of the at least one hypotonic shock is less than the duration of at least one of the at least two hypertonic shocks.

19. The method of claim 18, wherein the duration of the at least one hypotonic shock is less than or equal to about 30 minutes.

20. The method of claim 18, wherein the duration of the at least one hypotonic shock is in a range of about 2 minutes to about 30 minutes.

21. The method of claim 18, wherein the duration of the at least one hypotonic shock is in a range of about 5 minutes to about 20 minutes.

22. The method of any one of claims 1 and 4, wherein the duration of at least one of the at least two hypertonic shocks is in a range of about 2 minutes to about 30 minutes.

23. The method of any one of claims 1 and 4, wherein the microorganism comprises a microorganism selected from the group consisting of algae, bacteria, protists, yeasts, and fungi.

24. The method of claim 23, wherein the microorganism comprises algae.

25. The method of any one of claims 1 and 4, further comprising disrupting and/or extracting of the shocked microbial cells.

26. The method of claim 25, further comprising isolating of a cellular or intracellular constituent, metabolite, material for bioproduct from the disrupted and/or extracted microbial cells.

27. A method for enhancing disruption or extraction of algae, comprising:
obtaining algal cells, the algal cells having a cell membrane and having been grown in a source medium having a source tonicity with respect to said membrane;
suspending the algal cells, for a primary shock time period, in an initial aqueous suspension medium having a primary tonicity different from the source tonicity to provide primary shocked algal cells;
suspending the primary shocked algal cells, for a secondary shock time period, in an secondary aqueous suspension medium having a secondary tonicity different from the primary tonicity to provide secondary shocked algal cells;
suspending the secondary shocked algal cells, for a tertiary shock time period, in an tertiary aqueous suspension medium having a tertiary tonicity that is the same or different from the secondary tonicity to provide tertiary shocked algal cells; and
subjecting the tertiary shocked algal cells to a suitable disruption method, wherein the tonicity shocking comprises a synergistic combination of at least one hypotonic shock and at least two hypertonic shocks, and wherein the last tonicity shock is a hypertonic shock, wherein enhancing at least one of disruption and extraction of algal cells is afforded.

28. The method of claim 27, wherein the algae comprises at least one algal type selected from freshwater, brackish water, saltwater, and marine water algae.

29. A method for enhancing disruption or extraction of algae, comprising:
obtaining algal cells, the algal cells having a cell membrane and having been grown in a source medium having a source tonicity with respect to said membrane;
suspending the algal cells, for a primary shock time period, in an initial aqueous suspension medium having a primary tonicity different from the source tonicity to provide primary shocked algal cells;
suspending the primary shocked algal cells, for a secondary shock time period, in an secondary aqueous suspension medium having a secondary tonicity different from the primary tonicity to provide secondary shocked algal cells;
suspending the secondary shocked algal cells, for a tertiary shock time period, in an tertiary aqueous suspension medium having a tertiary tonicity that is the same or different from the secondary tonicity to provide tertiary shocked algal cells;
suspending the tertiary shocked microbial cells, for a quaternary shock time period, in an quaternary aqueous suspension medium having a quaternary tonicity that is different from the tertiary tonicity to provide quaternary shocked microbial cells; and
subjecting the quaternary shocked algal cells to a suitable disruption method, wherein the tonicity shocking comprises a synergistic combination of at least one hypotonic shock and at least two hypertonic shocks, and wherein the last tonicity shock is a hypertonic shock, wherein enhancing at least one of disruption and extraction of algal cells is afforded.

30. The method of claim 29, wherein the algae comprises at least one algal type selected from brackish water, saltwater, and marine water algae.

31. The method of any one of claims 27 and 29, further comprising disrupting and/or extracting of the shocked algal cells.

32. The method of claim 31, further comprising isolating a cellular constituent or bioproduct from the disrupted and/or extracted algal cells.

33. The method of claim 31, wherein the at least one hypotonic shock is of a duration less than or equal to 30 minutes.

34. The method of claim 33, further comprising isolating a cellular constituent or bioproduct from the disrupted and/or extracted algal cells.

35. The method of any one of claims 1, 4, 27 and 29, wherein a temperature below ambient temperature is used during the at least one hypotonic shock and/or during at least one of the at least two hypertonic shock steps.

36. The method of claims 35, wherein a temperature between about 0° C. and about 25° C. is used during the at least one hypotonic shock and/or during at least one of the at least two hypertonic shock steps.

37. The method of any one of claims 1 and 4, wherein the microbial cell comprise both algae and bacteria, and wherein the bacterial are differentially lysed during the synergistic osmotic shock steps.

38. The method of any one of claims 27 and 29, wherein the algal cells are present in combination with bacterial cells, and wherein the bacterial are differentially lysed during the synergistic osmotic shock steps.
